(19) United States
(12) Patent Application Publication
(10) Pub. No.: US 2024/0000905 A1
(43) Pub. Date: Jan. 4, 2024

(54) POLYMERIC NANOPARTICLE COMPOSITIONS FOR ENCAPSULATION AND SUSTAINED RELEASE OF NEUROMODULATORS

(71) Applicant: The Johns Hopkins University, Baltimore, MD (US)

(72) Inventors: Sashank Reddy, Baltimore, MD (US); Hai-Quan Mao, Baltimore, MD (US); Chenhu Qiu, Baltimore, MD (US)

(21) Appl. No.: 18/255,769

(22) PCT Filed: Nov. 30, 2021
(86) PCT No.: PCT/US2021/061174

§ 371 Date: Jun. 2, 2023

Related U.S. Application Data
(60) Provisional application No. 63/120,542, filed on Dec. 2, 2020.
(60) Provisional application No. 63/253,376, filed on Oct. 7, 2021.

(51) Int. Cl. A61K 38/48; A61K 9/51; A61K 9/00; A61P 21/02

(52) U.S. Cl. CPC A61K 38/4893; A61K 9/5146; B82Y 5/00; A61P 21/02; A61K 9/0019

(57) ABSTRACT

Nanoparticles or microgels comprising a polyelectrolyte nanocomplex comprising one or more neuromodulators, a carrier molecule, and a counter ion polymer, wherein the counter ion polymer has a charge enabling it to bind electrostatically to the one or more neuromodulators, methods of their preparation, and methods of treating a disease or condition are disclosed.

### BACKGROUND

Neuromodulators, including neurotoxins, are effective in both the aesthetic and therapeutic space. Neuromodulators are typically delivered via injection and paralyze muscle bodies with exceptional efficacy for a number of clinical indications, including providing relief for migraine headaches and reducing signs of aging for facial aesthetics. Despite a strong market performance for a combined $4.4 billion revenue in 2018, neuromodulator formulations currently on the market suffer from fast clearance from the injection site with only 14-day maximally effective release periods. Neuromodulator formulations known in the art therefore require reinjection at least every three months.

### SUMMARY

In some aspects, the presently disclosed subject matter provides a polyelectrolyte nanocomplex (PNC) comprising one or more neuromodulators, a carrier molecule, and a counter ion polymer, wherein the counter ion polymer has a charge enabling it to bind electrostatically to the one or more neuromodulators.

In some aspects, the presently disclosed subject matter provides a nanoparticle comprising the PNC and a non-water-soluble biodegradable polymer; wherein the polyelectrolyte nanocomplex (PNC) of one or more neuromodulators, the carrier molecule, and the counter ion polymer is distributed throughout the non-water-soluble biodegradable polymer. In such aspects, the nanoparticle is a sustained-release nanoparticle.

In some aspects, the one or more neuromodulators comprise a therapeutically active derivative of Clostridial neurotoxin. In certain aspects, the Clostridial neurotoxin comprises a therapeutically active derivative of a botulinum toxin. In certain aspects, the botulinum toxin is selected from the group consisting of therapeutically active derivatives of botulinum toxin types A, B, C, including C₁, D, E, F and G, and subtypes and mixtures thereof. In particular aspects, the one or more neuromodulators is selected from the group consisting of onabotulinumtoxin A, abobotulinumtoxin A, incobotulinumtoxin A, prabotulinumtoxin A, rimabotulinumtoxin B, and combinations thereof.

In some aspects, the carrier molecule comprises a polyelectrolyte selected from the group consisting of a cationic polymer, a protein, and a polysaccharide. In certain aspects, the protein is selected from the group consisting of IgG, collagen, gelatin, and serum albumin.

In some aspects, a weight ratio of the carrier molecule to the one or more neuromodulators can vary from about 1:1 to about 2000:1. In certain aspects, the weight ratio of the carrier molecule to the one or more neuromodulators is about 500:1.

In some aspects, the counter ion polymer is selected from the group consisting of dextran sulfate (DS), heparin (heparin sulfate), hyaluronic acid, and combinations thereof.

In some aspects, the biodegradable polymer is a copolymer selected from the group consisting of poly(L-lactic acid) (PLLA), polyglycolic acid (PGA), poly (D,L-lactic-co-glycolic acid) (PLGA), polycaprolactone (PCL), their PEGylated block copolymers, and combinations thereof. In certain aspects, the biodegradable polymer is selected from the group consisting of polyethylene glycol (PEG)-b-PLLA, PEG-b-PLGA, PEG-b-PCL, and combinations thereof. In particular aspects, the nanoparticle comprises one of: onabotulinumtoxinA (BoNTA):carrier protein:dextran sulfate (DS):PEG-b-PLGA in a m:1:1:n ratio, whereas m=0.0005 to 1, and n=3 to 10; (BoNTA+carrier):DS:PEG-b-PLGA is 1:1:5; or BoNTA:carrier is 1:1 to 1:2000.

In other aspects, the presently disclosed subject matter provides a process for generating a plurality of nanoparticles, the process comprising:
(a) forming a polyelectrolyte nanocomplex (PNC) by mixing a preformed solution of one or more neuromodulators and one or more carrier molecules and a counter ion polymer using a first continuous mixing process;
(b) co-precipitating the polyelectrolyte nanocomplex (PNC) with a biodegradable polymer using a second continuous mixing process; and
(c) forming a plurality of nanoparticles, wherein the polyelectrolyte nanocomplex (PNC) is distributed throughout the biodegradable polymer matrix.

In certain aspects, step (a) and step (b) proceed simultaneously. In certain aspects, the first continuous mixing process comprises a flash nanocomplexation (FNC) process. In certain aspects, the forming of the polyelectrolyte nanocomplex (PNC) is by electrostatic attraction between the one or more neuromodulators and the counter ion polymer. In certain aspects, the mixing of the polyelectrolyte nanocomplex (PNC) and the biodegradable polymer is by solvent-induced flash nanoprecipitation (FNP). In certain aspects, the forming of the nanoparticles occurs by the precipitation of the biodegradable polymer together with the polyelectrolyte nanocomplex (PNC).

In other aspects, the presently disclosed subject matter provides a process for generating a plurality of nanoparticles, the process comprising forming a polyelectrolyte nanocomplex (PNC) by mixing a preformed solution of one or more neuromodulators and one or more carrier molecules and a counter ion polymer using a continuous flash nanocomplexation (FNC) process.

In some aspects, the presently disclosed subject matter provides a method for preparing a neuromodulator-encapsulated polyelectrolyte nanocomplex (PNC), the method comprising: (a) preparing or providing an aqueous solution comprising one or more neuromodulators; (b) preparing or providing an aqueous solution of a carrier molecule; (c) mixing the aqueous solution of the neuromodulator and the aqueous solution of the carrier molecule to form a protein solution; and (d) mixing the protein solution with a counter ion polymer by a flash nanocomplexation (FNC) process to form a neuromodulator-encapsulated polyelectrolyte nanocomplex (PNC).

In other aspects, the presently disclosed subject matter provides a microgel comprising a nanoparticle or a polyelectrolyte nanocomplex (PNC) comprising one or more neuromodulators, a carrier molecule, and a counter ion polymer, wherein the counter ion polymer has a charge enabling it to bind electrostatically to the one or more neuromodulators; and a crosslinked hydrophilic polymer, wherein the nanoparticle or polyelectrolyte nanocomplex (PNC) is distributed throughout the crosslinked hydrophilic polymer.

In certain aspects, the crosslinked hydrophilic polymer comprises a hydrogel. In certain aspects, the hydrogel comprises a natural or synthetic hydrophilic polymer selected from the group consisting of hyaluronic acid, chitosan, heparin, alginate, fibrin, polyvinyl alcohol, polyethylene glycol, sodium polyacrylate, an acrylate polymers, and copolymers thereof. In particular aspects, the hydrogel comprises a crosslinked hyaluronic acid.

In certain aspects, the microgel comprises a plurality of microgel particles having a spherical or asymmetrical shape. In particular aspects, the plurality of microgel particles have a nominal size ranging from about 10 μm to about 1,000 μm. In yet more particular aspects, the microgel or the plurality of microgel polymers has a shear storage modulus from about 10 Pa to about 10,000 Pa.

In certain aspects, the microgel comprises a polyelectrolyte nanocomplex (PNC) having a nominal size ranging from about 20 nm to about 900 nm.

In other aspects, the microgel further comprising nanoparticle prepared from a biodegradable polymer. In certain aspects, the biodegradable polymer is selected from the group consisting of poly(L-lactic acid) (PLLA), polyglycolic acid (PGA), poly (D,L-lactic-co-glycolic acid) (PLGA), polycaprolactone (PCL), their PEGylated block copolymers, and combinations thereof. In particular aspects, the biodegradable polymer is selected from the group consisting of polyethylene glycol (PEG)-b-PLLA, PEG-b-PLGA, PEG-b-PCL, and combinations thereof. In certain aspects, the microgel comprises a nanoparticle having a nominal size ranging from about 20 nm to about 900 nm.

In some aspects, the crosslinked hydrophilic polymer further comprising one or more neuromodulators added directly thereto. In such aspects, the one or more neuromodulators added directly to the crosslinked hydrophilic polymer is a fraction of an amount of the one or more neuromodulators in the nanoparticle or polyelectrolyte nanocomplex (PNC). In particular aspects, the fraction of the one or more neuromodulators added directly to the crosslinked hydrophilic polymer has a range from about 0 to about 0.9.

In other aspects, the presently disclosed subject matter provides a process for generating a plurality of microgel particles, the process comprising:
(a) mixing a nanoparticle or polyelectrolyte nanocomplex (PNC) comprising one or more neuromodulators, a carrier molecule, and a counter ion polymer, and optionally a biodegradable polymer, with a hydrogel precursor;
(b) forming a hydrogel comprising the nanoparticle or polyelectrolyte nanocomplex (PNC) comprising one or more neuromodulators, a carrier molecule, and a counter ion polymer, and optionally a biodegradable polymer; and
(c) mechanically breaking the hydrogel into a plurality of microgel particles.

In certain aspects, the plurality of microgel particles has a nominal size ranging from about 10 μm to 1,000 μm.

In other aspects, the presently disclosed subject matter provides a method for treating a disease or condition, the method comprising administering a presently disclosed nanoparticle or microgel to a subject in treat of treatment thereof. In certain aspects, the disease or condition is selected from the group consisting of a cosmetic condition, focal dystonias, cervical dystonia (CD), chronic sialorrhea, and muscle spasticity. In particular aspects, the muscle spasticity is related to an overactive muscle movement selected from the group consisting of cerebral palsy, post-stroke spasticity, post-spinal cord injury spasticity, spasms of the head and neck, eyelid, vagina, limbs, jaw, and vocal cords, clenching of muscles associated with muscles of the esophagus, jaw, lower urinary tract and bladder, and anus, and refractory overactive bladder. In certain aspects, the disease or condition comprises muscle disorder selected from the group consisting of strabismus, blepharospasm, hemifacial spasm, infantile esotropia, restricted ankle motion due to lower-limb spasticity associated with stroke in adults, and lower-limb spasticity in pediatric patients two years of age and older. In particular aspects, the disease or condition comprises excessive sweating. In certain aspects, the disease or condition is selected from the group consisting of a headache, a migraine headache, neuropathic pain, chronic pain, osteoarthritis pain, arthritic pain, allergy symptoms, depression, and premature ejaculation.

In certain aspects, the method comprises administering two or more formulations of the nanoparticle or microgel, wherein the two or more formulations of the nanoparticle or microgel each have a different release profile.

In other aspects, the presently disclosed subject matter provides a pharmaceutical composition comprising a presently disclosed nanoparticle or microgel and a pharmaceutically acceptable carrier. In yet other aspects, the presently disclosed subject matter provides a kit comprising a presently disclosed nanoparticle and/or microgel.

In other aspects, the presently disclosed subject matter provides a sustained release formulation comprising the presently disclosed nanoparticle or microgel, wherein the formulation provides an effective concentration of the one or more neuromodulators in soft tissue for a period of time between about 3 days to about 200 days.

In other aspects, the presently disclosed method for treating a disease or condition, the method comprising administering a sustained release formulation comprising the presently disclosed nanoparticle or microgel, the method comprising local administration by injection of the sustained release formulation, wherein the one or more neuromodulators is released from the sustained release formulation over a period of time from about 3 days to about 200 days, thereby treating a disease or condition.

In certain aspects, the disease or condition is selected from the group consisting of a cosmetic condition, focal dystonias, cervical dystonia (CD), chronic sialorrhea, and muscle spasticity.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

Having thus described the presently disclosed subject matter in general terms, reference will now be made to the accompanying Figures, which are not necessarily drawn to scale, and wherein:

FIG. 1 is a schematic illustration of the presently disclosed two-step (FNC-FNP) preparation process of nanoparticles with encapsulated polyelectrolyte nanocomplex (PNC) of a protein therapeutic and a counter ion polyelectrolyte. A three-inlet device is shown here for the second step. It is possible to switch this to a two-inlet or a four-inlet mixing chamber based on the specific requirements for solvent exchange in the FNP process (from International PCT Patent Application Publication No. WO/2019/148147, to Mao et al., for “Polymeric nanoparticle compositions for encapsulation and sustained release of protein therapeutics, published Aug. 1, 2019);

FIG. 2 is a schematic illustration of single-step encapsulation of protein therapeutics using a four-inlet multi-inlet vortex mixer. Polyelectrolyte nanocomplex (PNC) co-precipitates with biodegradable polyester forming a nanoparticle, and distributes throughout the polymer nanoparticle (from International PCT Patent Application Publication No. WO/2019/148147, to Mao et al., for “Polymeric nanoparticle compositions for encapsulation and sustained release of protein therapeutics, published Aug. 1, 2019);

FIG. 3 is a representative TEM image of botulinum toxin A (BoNTA) nanoparticles;

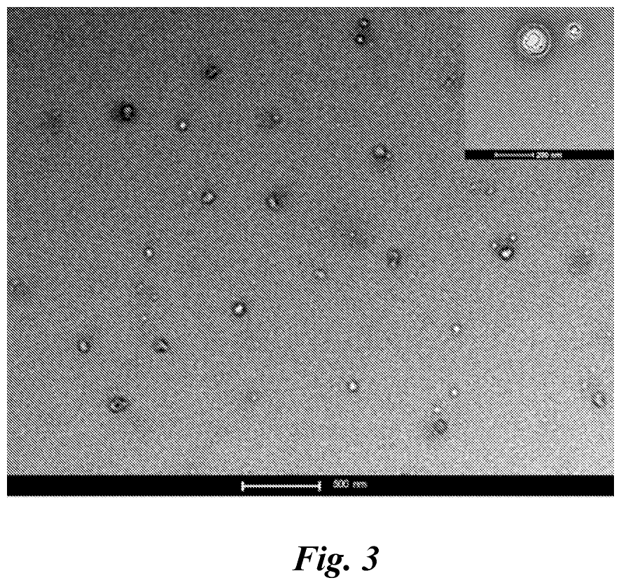

FIG. 4 demonstrates that the presently disclosed NanoTox formulation delivers a near-linear release of protein with a high degree of bioactivity retention;

FIG. 5 demonstrates that BoNTA released from the presently disclosed NanoTox formulation retains >80% bioactivity within 28 days;

FIG. 6 is a representative dynamic light scattering (DLS) graph showing the size distribution of the presently disclosed BoNTA/dextran sulfate (DS) polyelectrolyte nanocomplex (PNC) formulation (referred to herein as “NP4”), in addition to the assessment data on zeta potential and polydispersity index (PDI) of the NP4, which were produced with 2 mg/mL of BoNTA and human serum albumin (HSA) at a ratio of 1:500 and 2 mg/mL of dextran sulfate (DS) at a flow rate of 10 mL/min;

FIG. 7 demonstrates that BoNTA is released from the presently disclosed BoNTA/DS polyelectrolyte nanocomplexes (PNCs, NP4) measured by ELISA, releasing 87% of BoNTA within 3 days;

FIG. 8 is an in vitro release profile of BoNTA from microgel particle formulation 1 (MP1) in PBS at 37° C.;

FIG. 9 shows the in vitro bioactivity of released BoNTA from MP1 formulation;

FIG. 10 is an in vitro release profile of BoNTA from microgel particle formulation 2 (MP2);

FIG. 11 shows the in vitro bioactivity of the released BoNTA samples from MP2;

FIG. 12 is an in vitro release profile of BoNTA from microgel particle formulation 3 (MP3); and

FIG. 13 shows in vivo functional data (stimulated grip strength recovery) after i.m. injection of different NanoTox formulations in comparison with free BoNTA injections.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### Polymeric Nanoparticle and Polyelectrolyte Nanocomplex Compositions for Encapsulation and Sustained Release of Neuromodulators

The presently disclosed subject matter provides a platform for delivering one or more neuromodulators, including neurotoxins, to a target site. This delivery platform provides a tunable, sustained release profile and high payload capacity of the neuromodulator, while also allowing for high retention of its bioactivity. The platform utilizes a proven scalable, highly translational manufacturing process that enables continuous particle production with a high yield under cGMP conditions. This combination provides novel engineered biodegradable nanoparticles with a rapid micro-mixing process to encapsulate one or more neuromodulators, including neurotoxins, within a biodegradable polymer.

The presently disclosed subject matter enables high neuromodulator payload capacity and high encapsulation efficiency due, in part, to a flash micro-mixing process to generate nanoparticles under a super-saturation condition. Nanoparticles formed under these conditions offer a sustained and prolonged release of one or more neuromodulators over an extended period of time.

Previously reported nanoparticles for encapsulating proteins either release the payload rapidly or achieve prolonged presence through surface conjugation, which limits loading capacity and increases susceptibility to protein loss via surface erosion. In contrast, the presently disclosed processes ensure completion of the nanoparticle assembly before the equilibrium partition and protein unfolding, thus achieving high level of preservation of bioactivity and stability during release and storage.

The presently disclosed manufacturing processes also offer a high level of uniformity of the assembly process, a high quality of the nanoparticles produced, and is highly scalable. See U.S. Pat. No. 10,441,549 to Mao et al., for “Methods of preparing polyelectrolyte complex nanoparticles,” issued Oct. 15, 2019, and International PCT Patent Application Publication No. WO/2019/148147, to Mao et al., for “Polymeric nanoparticle compositions for encapsulation and sustained release of protein therapeutics, published Aug. 1, 2019, each of which is incorporated herein in its entirety.

Neuromodulator-polyanion polyelectrolyte nanocomplex (PNC) is critical for bioactivity retention and regulating release rate of the protein. Without such polyelectrolyte nanocomplex (PNC), it is not possible to load protein at a high encapsulation efficiency and loading level and to yield a sustained release profile.

Uniform distribution is achieved as a result of the unique assembly process (kinetically controlled heterogeneous assembly). Uniform distribution also is critical to achieve long-term sustained release of the protein and to enable loading of different proteins (e.g., carrier proteins) at predetermined ratios with high level of control.

### A. Polyelectrolyte Nanocomplex (PNC) or Nanoparticle Comprising One or More Neuromodulators, a Carrier Molecule, and a Counter Ion Polymer Distributed Throughout a Biodegradable Polymer

In some embodiments, the presently disclosed subject matter provides a polyelectrolyte nanocomplex (PNC) comprising one or more neuromodulators, a carrier molecule, and a counter ion polymer, wherein the counter ion polymer has a charge enabling it to bind electrostatically to the one or more neuromodulators.

In some embodiments, the presently disclosed subject matter provides a nanoparticle comprising the polyelectrolyte nanocomplex (PNC) and a non-water-soluble biodegradable polymer, wherein the polyelectrolyte nanocomplex (PNC) is distributed throughout the non-water-soluble biodegradable polymer.

In certain embodiments, the nanoparticle is a sustained-release nanoparticle comprising a polyelectrolyte nanocomplex (PNC) comprising one or more neuromodulators, a carrier molecule, and a counter ion polymer having a charge enabling it to bind electrostatically to the one or more neuromodulators and the carrier molecule; and a non-water-soluble biodegradable polymer; wherein the polyelectrolyte nanocomplex (PNC) comprising one or more neuromodulators, the carrier molecule, and the counter ion polymer is distributed throughout the non-water-soluble biodegradable polymer.

In some embodiments, the one or more neuromodulators comprise a therapeutically active derivative of Clostridial neurotoxin. In some embodiments, the Clostridial neurotoxin comprises a therapeutically active neurotoxin derived from Clostridium botulinum, a Gram-positive, rod-shaped, anaerobic, spore-forming, motile bacterium with the ability to produce the neurotoxin botulinum. The botulinum toxin can induce flaccid paralysis in humans, which is characterized by weakness, paralysis and reduced muscle tone. In some embodiments, the one or more neuromodulators comprise a therapeutically active derivative of a botulinum toxin.

In some embodiments, the botulinum toxin is selected from the group consisting of therapeutically active derivatives of botulinum toxin types A, B, C, including C₁, D, E, F and G, and subtypes and mixtures thereof. See for example, U.S. Pat. No. 8,501,187 B2, which is incorporated herein by reference in its entirety.

As used herein, “Botulinum toxin” means a neurotoxin produced by Clostridium botulinum, as well as a botulinum toxin (or the light chain or the heavy chain thereof) made recombinantly by a non-Clostridial species. The term “botulinum toxin”, as used herein, encompasses the botulinum toxin serotypes A, B, C, D, E, F and G, and their subtypes and any other types of subtypes thereof, or any re-engineered proteins, analogs, derivatives, homologs, parts, sub-parts, variants, or versions, in each case, of any of the foregoing.

“Botulinum toxin”, as used herein, also encompasses a “modified botulinum toxin”. Further “botulinum toxin” as used herein also encompasses a botulinum toxin complex, (for example, the 300, 600 and 900 kDa complexes), as well as the neurotoxic component of the botulinum toxin (150 kDa) that is unassociated with the complex proteins.

“Clostridial derivative” refers to a molecule which contains any part of a clostridial toxin. As used herein, the term “clostridial derivative” encompasses native or recombinant neurotoxins, recombinant modified toxins, fragments thereof, a Targeted vesicular Exocytosis Modulator (TEM), or combinations thereof.

“Clostridial toxin” refers to any toxin produced by a Clostridial toxin strain that can execute the overall cellular mechanism whereby a Clostridial toxin intoxicates a cell and encompasses the binding of a Clostridial toxin to a low or high affinity Clostridial toxin receptor, the internalization of the toxin/receptor complex, the translocation of the Clostridial toxin light chain into the cytoplasm and the enzymatic modification of a Clostridial toxin substrate.

In some embodiments, the botulinum toxin can be a recombinant botulinum neurotoxin, such as botulinum toxins produced by E. coli. In some embodiments, the botulinum neurotoxin can be a modified neurotoxin, that is a botulinum neurotoxin which has at least one of its amino acids deleted, modified or replaced, as compared to a native toxin, or the modified botulinum neurotoxin can be a recombinant produced botulinum neurotoxin or a derivative or fragment thereof. In certain embodiments, the modified toxin has an altered cell targeting capability for a neuronal or non-neuronal cell of interest. This altered capability is achieved by replacing the naturally-occurring targeting domain of a botulinum toxin with a targeting domain showing a selective binding activity for a non-botulinum toxin receptor present in a non-botulinum toxin target cell. Such modifications to a targeting domain result in a modified toxin that is able to selectively bind to a non-botulinum toxin receptor (target receptor) present on a non-botulinum toxin target cell (re-targeted). A modified botulinum toxin with a targeting activity for a non-botulinum toxin target cell can bind to a receptor present on the non-botulinum toxin target cell, translocate into the cytoplasm, and exert its proteolytic effect on the SNARE complex of the target cell. In essence, a botulinum toxin light chain comprising an enzymatic domain is intracellularly delivered to any desired cell by selecting the appropriate targeting domain.

In some embodiments, the botulinum toxin comprises a modified botulinum toxin comprising a natural heavy chain and a modified light chain. See, for example, U.S. Pat. No. 9,186,396 to Frevert et al. for PEGylated mutated Clostridium botulinum toxin, issued Nov. 17, 2015; U.S. Pat. No. 8,912,140 to Frevert et al. for PEGylated mutated Clostridium botulinum toxin, issued Dec. 16, 2014; U.S. Pat. No. 8,298,550 to Frevert et al. for PEGylated mutated Clostridium botulinum toxin, issued Oct. 30, 2012; U.S. Pat. No. 8,003,601 for Frevert et al. for Pegylated mutated Clostridium botulinum toxin, issued Aug. 23, 2011.

In some embodiments, the one or more neuromodulator comprises a botulinum neurotoxin that is altered with regard to their protein structure in comparison to the corresponding wild-type neurotoxins. See, e.g., U.S. Pat. No. 8,748,151 to Frevert for Clostridial neurotoxins with altered persistency, issued Jun. 10, 2014.

Fermentation processes for preparing botulinum toxins are known in the art. See, e.g., Methods of preparing U.S. Pat. No. 7,927,836 to Doelle et al. for Device and method for the production of biologically active compounds by fermentation, issued Apr. 19, 2011; U.S. Pat. No. 10,465,178 to Ton et al. for Process and system for obtaining botulinum neurotoxin, issued Nov. 5, 2019.

Highly pure botulinum toxins can be prepared by cultivating Clostridium botulinum under conditions that allow production of a botulinum toxin and then isolating the neurotoxic component from the botulinum toxin. See U.S. Pat. No. 10,653,754 to Pfeil et al., Highly pure neurotoxic component of a botulinum toxin and uses thereof, issued May 19, 2020 (providing neurotoxins having a single-chain content of less than 1.70 wt. %, and a total purity of at least 99.90 wt. %); U.S. Pat. No. 9,937,245 to Pfeil et al. for Highly pure neurotoxic component of a botulinum toxin, process for preparing same, and uses thereof, issued Apr. 10, 2018.

Representative commercial neuromodulators include, but are not limited to, botulinum toxin A, such as onabotulinumtoxinA (BOTOX® (Allergan, Inc.)), abobotulinumtoxinA (DYSPORT® and AZZALURE® (Galderma Laboratories, L.P.)), incobotulinumtoxinA (IPSEN®, XEOMIN®, and BOCOUTURE® (Pharma GmbH & Co. KGaA)), and prabotulinumtoxinA (JEUVEAU® (Evolu (manufactured by Daewoong))), BTX-A (Lontox and Prosigne (Lanzhou Institute of Biological Products) and Neuronox (MedyTox, Inc.)) and botulinum toxin B, such as rimabotulinumtoxinB (MYOBLOC® and NEUROBLOC® (Solstice Neurosciences, Inc)).

Accordingly, in some embodiments, the one or more neuromodulators can be selected from the group consisting of onabotulinumtoxin A, abobotulinumtoxin A, incobotulinumtoxin A, prabotulinumtoxin A, rimabotulinumtoxin B, and combinations thereof.

Neuromodulators, such as the botulinum toxins, are potent enough to require administration of a minute amount of functional protein. It is very difficult, however, to load the therapeutically active neuromodulator directly without the use of a carrier molecule. Therefore, formation of neuromodulator-polyanion nanocomplexes is critical for regulating the release of the neuromodulator and retention of its bioactivity. Without the formation of neuromodulator-polyanion nanocomplexes it is not possible to load that protein at a high encapsulation efficiency and loading level. Further, it is not possible to yield a sustained release profile as disclosed herein.

Polyelectrolytes, including synthetic polymers, proteins, and polysaccharides, with the same net charge as the neuromodulator can serve the role of a carrier. Proteins are natural choice due to the similarity of structure and charge density between the carrier protein and neuromodulator. Representative proteins suitable for use as carriers in the presently disclosed formulations include, but are not limited to, IgG, collagen, gelatin, and serum albumin, including human serum albumin, and mouse serum albumin, and combinations thereof. In particular embodiments, the carrier molecule comprises serum albumin.

Other cationic polymers suitable for use with the presently disclosed compositions and methods include, but are not limited to, chitosan, PAMAM dendrimers, polyethylenimine (PEI), protamine, poly(arginine), poly(lysine), poly(beta-aminoesters), and cationic peptides and derivatives thereof.

Different proteins having a wider range of isoelectric points (e.g., from about 4.5 to about 11) can be encapsulated into such a formulation. In some embodiments, the one or more neuromodulators and carriers selected for the presently disclosed formulations have isoelectric points in the range of about 5.0 to about 8.0, including an isoelectric point of about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, and 8.0.

The weight ratio of carrier to neuromodulator can vary from about 1:1 to about 2000:1, including a weight ratio of carrier to neuromodulator of about 1:1, 2:1, 3:1, 4:1, 5:1, 6:1, 7:1, 8:1, 9:1, 10:1, 15:1, 20:1, 25:1, 30:1, 35:1, 40:1, 45:1, 50:1, 55:1, 60:1, 65:1, 70:1, 75:1, 80:1, 85:1, 90:1, 95:1, 100:1, 110:1, 115:1, 120:1, 125:1, 130:1, 135:1, 140:1, 145:1, 150:1, 155:1, 160:1, 165:1, 170:1, 175:1, 180:1, 185:1, 190:1, 195:1, 200:1, 210:1, 215:1, 220:1, 225:1, 230:1, 235:1, 240:1, 245:1, 250:1, 255:1, 260:1, 265:1, 270:1, 275:1, 280:1, 285:1, 290:1, 295:1, 300:1, 310:1, 315:1, 320:1, 325:1, 330:1, 335:1, 340:1, 345:1, 350:1, 355:1, 360:1, 365:1, 370:1, 375:1, 380:1, 385:1, 390:1, 395:1, 400:1, 410:1, 415:1, 420:1, 425:1, 430:1, 435:1, 440:1, 445:1, 450:1, 455:1, 460:1, 465:1, 470:1, 475:1, 480:1, 485:1, 490:1, 495:1, 500:1, 510:1, 515:1, 520:1, 525:1, 530:1, 535:1, 540:1, 545:1, 550:1, 555:1, 560:1, 565:1, 570:1, 575:1, 580:1, 585:1, 590:1, 595:1, 600:1, 610:1, 615:1, 620:1, 625:1, 630:1, 635:1, 640:1, 645:1, 650:1, 655:1, 660:1, 665:1, 670:1, 675:1, 680:1, 685:1, 690:1, 695:1, 700:1, 710:1, 715:1, 720:1, 725:1, 730:1, 735:1, 740:1, 745:1, 750:1, 755:1, 760:1, 765:1, 770:1, 775:1, 780:1, 785:1, 790:1, 795:1, 800:1, 810:1, 815:1, 820:1, 825:1, 830:1, 835:1, 840:1, 845:1, 850:1, 855:1, 860:1, 865:1, 870:1, 875:1, 880:1, 885:1, 890:1, 895:1, 900:1, 910:1, 915:1, 920:1, 925:1, 930:1, 935:1, 940:1, 945:1, 950:1, 955:1, 960:1, 965:1, 970:1, 975:1, 980:1, 985:1, 990:1, 995:1, 1000:1, 1010:1, 1015:1, 1020:1, 1025:1, 1030:1, 1035:1, 1040:1, 1045:1, 1050:1, 1055:1, 1060:1, 1065:1, 1070:1, 1075:1, 1080:1, 1085:1, 1090:1, 1095:1, 1100:1, 1110:1, 1115:1, 1120:1, 1125:1, 1130:1, 1135:1, 1140:1, 1145:1, 1150:1, 1155:1, 1160:1, 1165:1, 1170:1, 1175:1, 1180:1, 1185:1, 1190:1, 1195:1, 1200:1, 1210:1, 1215:1, 1220:1, 1225:1, 1230:1, 1235:1, 1240:1, 1245:1, 1250:1, 1255:1, 1260:1, 1265:1, 1270:1, 1275:1, 1280:1, 1285:1, 1290:1, 1295:1, 1300:1, 1310:1, 1315:1, 1320:1, 1325:1, 1330:1, 1335:1, 1340:1, 1345:1, 1350:1, 1355:1, 1360:1, 1365:1, 1370:1, 1375:1, 1380:1, 1385:1, 1390:1, 1395:1, 1400:1, 1410:1, 1415:1, 1420:1, 1425:1, 1430:1, 1435:1, 1440:1, 1445:1, 1450:1, 1455:1, 1460:1, 1465:1, 1470:1, 1475:1, 1480:1, 1485:1, 1490:1, 1495:1, 1500:1, 1510:1, 1515:1, 1520:1, 1525:1, 1530:1, 1535:1, 1540:1, 1545:1, 1550:1, 1555:1, 1560:1, 1565:1, 1570:1, 1575:1, 1580:1, 1585:1, 1590:1, 1595:1, 1600:1, 1610:1, 1615:1, 1620:1, 1625:1, 1630:1, 1635:1, 1640:1, 1645:1, 1650:1, 1655:1, 1660:1, 1665:1, 1670:1, 1675:1, 1680:1, 1685:1, 1690:1, 1695:1, 1700:1, 1710:1, 1715:1, 1720:1, 1725:1, 1730:1, 1735:1, 1740:1, 1745:1, 1750:1, 1755:1, 1760:1, 1765:1, 1770:1, 1775:1, 1780:1, 1785:1, 1790:1, 1795:1, 1800:1, 1810:1, 1815:1, 1820:1, 1825:1, 1830:1, 1835:1, 1840:1, 1845:1, 1850:1, 1855:1, 1860:1, 1865:1, 1870:1, 1875:1, 1880:1, 1885:1, 1890:1, 1895:1, 1900:1, 1910:1, 1915:1, 1920:1, 1925:1, 1930:1, 1935:1, 1940:1, 1945:1, 1950:1, 1955:1, 1960:1, 1965:1, 1970:1, 1975:1, 1980:1, 1985:1, 1990:1, 1995:1, and 2000:1.

In some embodiments, the weight ratio of the carrier protein to the neuromodulator is about 500:1.

As used herein, the term “counter ion polymer” includes a polymer having a charge so that the polymer is able to bind electrostatically to the one or more neuromodulators. Examples include a protein that is net positively charged the binds to a counter ion polymer that has a net negative charge or vice versa.

In some embodiments, the counter ion polymer is negatively charged. In particular embodiments, the counter ion polymer is selected from the group consisting of dextran sulfate (DS), heparin (heparin sulfate), hyaluronic acid, and combinations thereof.

Other anionic polymers suitable for use with the presently disclosed compositions and methods include, but are not limited to, poly(aspartic acid), poly(glutamic acid), negatively charged block copolymers, alginate, tripolyphosphate (TPP), and oligo (glutamic acid).

In some embodiments, the biodegradable polymer is a copolymer selected from the group consisting of poly(L-lactic acid) (PLLA), polyglycolic acid (PGA), poly(D,L-lactic-co-glycolic acid) (PLGA), polycaprolactone (PCL), their PEGylated block copolymers, and combinations thereof. In particular embodiments, the biodegradable polymer is selected from the group consisting of polyethylene glycol (PEG)-b-PLLA, PEG-b-PLGA, PEG-b-PCL, and combinations thereof. In some embodiments, the presently disclosed formulation comprises one of: BoNTA:dextran sulfate (DS):PEG-b-PLGA in a m:1:1:n ratio, whereas m=0.0005 to 1, and n=3 to 10; (BoNTA+carrier):DS:PEG-b-PLGA is 1:1:5; or BoNTA:carrier is 1:1 to 1:2000.

In some embodiments, the nanoparticles range in size from about 20 nm to about 500 nm in diameter, including about 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, and 500 nm. For example, in some embodiments, the present nanoparticles have an average particle size of less than about 500 nm, less than about 400 nm, less than about 300 nm, less than about 200 nm, and less than about 100 nm (homogenous diameter). In some embodiments, the nanoparticles have an average particle size of approximately 100 nm.

In some embodiments, the nanoparticles have a polydispersity index lower than about 0.3. In certain embodiments, the nanoparticles have a polydispersity index ranging from about 0.05 to about 0.3, including a polydispersity index of about 0.05, 0.06, 0.07, 0.08, 0.09, 0.1, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, and 0.30.

### B. Methods for Preparing a Nanoparticle Comprising One or More Neuromodulators, a Carrier Molecule, and a Counter Ion Polymer Distributed Throughout a Biodegradable Polymer

In other embodiments, the presently disclosed subject matter provides a process for generating a plurality of nanoparticles, the process comprising:
(a) forming a polyelectrolyte nanocomplex (PNC) by mixing a preformed solution of one or more neuromodulators and one or more carrier molecules and a counter ion polymer using a first continuous mixing process;
(b) co-precipitating the polyelectrolyte nanocomplex (PNC) with a biodegradable polymer using a second continuous mixing process; and
(c) forming a plurality of nanoparticles, wherein the polyelectrolyte nanocomplex (PNC) is distributed throughout the biodegradable polymer matrix.

In some embodiments, step (a) and step (b) proceed simultaneously.

In some embodiments, the first continuous mixing process comprises a flash nanocomplexation (FNC) process. The FNC process is described in U.S. Pat. No. 10,441,549 to Mao et al., for Methods of preparing polyelectrolyte complex nanoparticles, issued Oct. 15, 2019, which is incorporated herein by reference in its entirety.

In other embodiments, the presently disclosed subject matter provides a process for generating a plurality of nanoparticles, the process comprising forming a polyelectrolyte nanocomplex (PNC) by mixing a preformed solution of one or more neuromodulators and one or more carrier molecules and a counter ion polymer using a continuous flash nanocomplexation (FNC) process.

As used herein, the term “polyelectrolyte nanocomplexes (PNCs)” (also known as polyelectrolyte coacervates) are the association complexes with size ranging from 20 to 900 nm, formed between oppositely charged polymers (e.g., polymer-polymer, polymer-drug, and polymer-drug-polymer). Polyelectrolyte nanocomplexes (PNCs) are formed due to electrostatic interaction between oppositely charged polyions, i.e. water-soluble polycations and water-soluble polyanions. As used herein, the term “water-soluble” refers to the ability of a compound to be able to be dissolved in water. As used herein, the terms “continuous” or “continuously” refer to a process that is uninterrupted in time, such as the generation of polyelectrolyte nanocomplex (PNC) while at least two presently disclosed streams are flowing into a confined chamber.

In some embodiments, the forming of the polyelectrolyte nanocomplex (PNC) is by electrostatic attraction between the one or more neuromodulators and the counter ion polymer.

In some embodiments, the mixing of the polyelectrolyte nanocomplex (PNC) and the biodegradable polymer is by solvent-induced flash nanoprecipitation (FNP).

Flash nanoprecipitation (FNP) offers a continuous and scalable process that has been used for the production of block copolymer nanoparticles. Flash nanoprecipitation (FNP) uses a kinetic controlled process to generate nanoparticles in a continuous and scalable manner by using confined impinging jet (CIJ) or multi-inlet vortex mixer (MIVM) device. The rapid micromixing conditions of FNP (on the order of 1 msec) establishes homogeneous supersaturation conditions and controlled precipitation of hydrophobic solutes (organic or inorganic) using block copolymer self-assembly. Compared to bulk preparation methods, the FNP process allows for the formation of uniform aggregates with tunable size in a continuous flow operation process, which is amenable for scale-up production. This process also offers a higher degree of versatility and control over particle size and distribution, higher drug encapsulation efficiency, and improved colloidal stability.

In some embodiments, the forming of the nanoparticles occurs by the precipitation of the biodegradable polymer together with the polyelectrolyte nanocomplex (PNC).

In particular embodiments, polyelectrolyte nanocomplex (PNC) comprising one or more neuromodulators, one or more carrier molecules, and one or more a counter ion polymers are generated through flash nanocomplexation (FNC), and then co-precipitated with one or more biodegradable polymers in an FNP solvent exchange process.

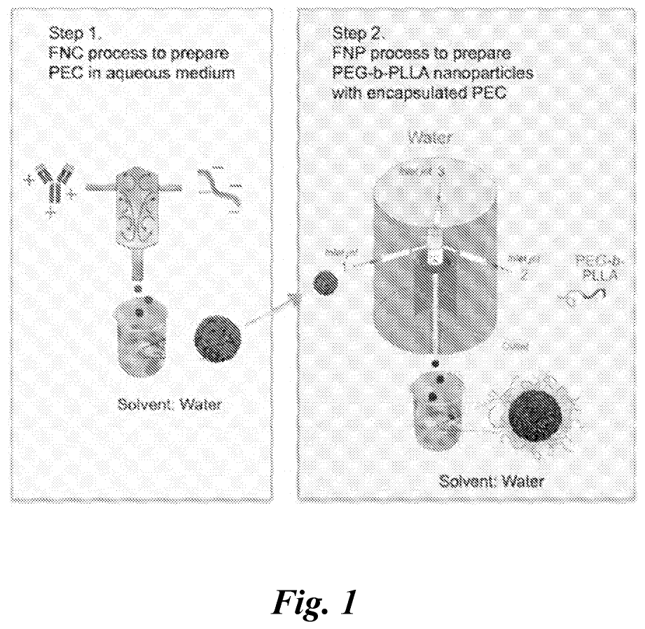

This two-step process for forming PNC-containing nanoparticles is provided in FIG. 1 (from International PCT Patent Application Publication No. WO/2019/148147, to Mao et al., for “Polymeric nanoparticle compositions for encapsulation and sustained release of protein therapeutics, published Aug. 1, 2019). In this process, the polycation solution (i.e., the solution comprising the one or more neuromodulators), polyanion solution (i.e., one or more counter ion polymers, e.g., dextran sulfate, heparin sulfate, and the like), and block copolymer dissolved in a water miscible solvent are introduced into a defined chamber at an optimized set of flow rates to achieve efficient mixing, therefore obtaining nanoparticles with efficient loading of the one or more neuromodulators.

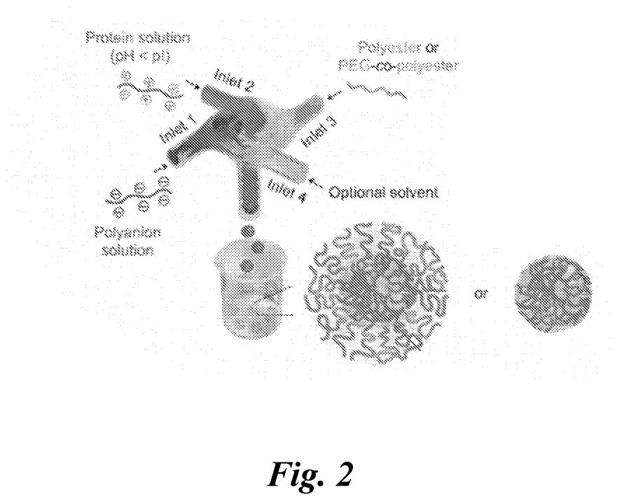

In certain embodiments, the two processes of polyelectrolyte nanocomplex complexation (by the FNC process) and polymer nanoparticle formation as a result of flash nanoprecipitation (FNP) are combined in a single-step phase separation process. This process involves continuously infusing solution jets of: (1) one or more neuromodulators dissolved in an aqueous solvent at a pH that is lower than the isoelectric point (pi) of the protein; (2) a polyanion, e.g. dextran sulfate (DS), heparin (heparin sulfate) and hyaluronic acid, and the like, dissolved in an aqueous solvent; (3) a biodegradable polymer dissolved in a water-miscible organic solvent; and (4) an additional solvent jet to maintain achieve a specific solvent polarity to induce efficient phase separation and nanoparticle formation at a set of predetermined flow rates through a confined impinging jet mixer or a multi-inlet vortex mixer, resulting in the formation of polyelectrolyte nanocomplex (PNC)-containing nanoparticles. A representative embodiments for performing a single-step encapsulation of protein therapeutics using a four-inlet multi-inlet vortex mixer is provided in FIG. 2. (from International PCT Patent Application Publication No. WO/2019/148147, to Mao et al., for “Polymeric nanoparticle compositions for encapsulation and sustained release of protein therapeutics, published Aug. 1, 2019).

In some embodiments, the water-miscible organic solvent is selected from the group consisting of acetyl nitrile (ACN), dimethyl sulfoxide (DMSO), tetrahydrofuran (THF), dimethylformamide (DMF), ethanol, isopropyl alcohol (IPA), hexafluoroisopropanol (HFIP), and combinations thereof.

The presently disclosed methods produce nanoparticles comprising a monolithic matrix comprising the biodegradable polymer with the polyelectrolyte nanocomplex (PNC) including the one or more neuromodulators distributed throughout the biodegradable polymer matrix. The presently disclosed process results in nanoparticles capable of having a wider range of loading capacity. In this process, discrete polyelectrolyte nanocomplex (PNC) is encapsulated in the hydrophobic polymer nanoparticle, where the polyelectrolyte nanocomplex (PNC) serves as a nucleus co-precipitated with a hydrophobic polymer, resulting in a structure of a multi-core matrix nanoparticle with polyelectrolyte nanocomplex (PNC) uniformly distributed throughout the core. More specifically, in the single-step process, the polyelectrolyte nanocomplex (PNC) forms instantaneously and serves as the nucleus to induce co-precipitation of hydrophobic polymer nanoparticle, again yielding uniform distribution of the polyelectrolyte nanocomplex (PNC) throughout the nanoparticle.

In some embodiments, the plurality of nanoparticles have a Z-average particle size of about 20 nm to about 900 nm, including about 20, 30, 40, 50, 60, 70, 80, 90, 100, 200, 300, 400, 500, 600, 700, 800, and 900 nm, and with a size distribution (PDI) of about 0.1 to about 0.4, including about 0.1, 0.2, 0.3, and 0.4.

In some embodiments, the plurality of nanoparticles have a negative surface charge with an average zeta potential of about −10 mV to about −35 mV, including about −10, −15, −20, −25, −30, and −35 mV.

In some embodiments, the plurality of nanoparticles have an encapsulation efficiency of about 60% to about 95%, including about 60, 65, 70, 75, 80, 85, 90, and 95% encapsulation efficiency.

In some embodiments, the plurality of nanoparticles have a loading level of about 2% to about 50%, including about 2, 5, 10, 15, 20, 25, 30, 35, 40, 45, and 50% loading level.

In some embodiments, the plurality of nanoparticles have a release duration of about 7 days to about 180 days, including about 7, 8, 9, 10, 20, 30, 40, 50, 60, 70, 80, 90, 100, 110, 120, 130, 140, 150, 160, 170, and 180 days.

### C. Method for Preparing a Neuromodulator-Encapsulated Polyelectrolyte Nanocomplex (PNC)

In some embodiments, the presently disclosed subject matter provides a method for preparing a neuromodulator-encapsulated polyelectrolyte nanocomplex (PNC), the method comprising:
(a) preparing or providing an aqueous solution comprising one or more neuromodulators;
(b) preparing or providing an aqueous solution of a carrier molecule;
(c) mixing the aqueous solution of the neuromodulator and the aqueous solution of the carrier molecule to form a protein solution; and
(d) mixing the protein solution with a counter ion polymer by a flash nanocomplexation (FNC) process to form a neuromodulator-encapsulated polyelectrolyte nanocomplex (PNC).

In some embodiments, the one or more neuromodulators comprise a therapeutically active derivative of Clostridial neurotoxin. In certain embodiments, the Clostridial neurotoxin comprises a therapeutically active derivative of a botulinum toxin. In particular embodiments, the botulinum toxin is selected from the group consisting of therapeutically active derivatives of botulinum toxin types A, B, C, including C₁, D, E, F and G, and subtypes and mixtures thereof. In particular embodiments, the one or more neuromodulators is selected from the group consisting of onabotulinumtoxin A, abobotulinumtoxin A, incobotulinumtoxin A, prabotulinumtoxin A, rimabotulinumtoxin B, and combinations thereof.

In some embodiments, the carrier molecule comprises a polyelectrolyte selected from the group consisting of a cationic polymer, a protein, and a polysaccharide. In some embodiments, the protein is selected from the group consisting of IgG, collagen, gelatin, and serum albumin.

In some embodiments, a weight ratio of the carrier molecule to the one or more neuromodulators can vary from about 1:1 to about 2000:1. In particular embodiments, the weight ratio of the carrier molecule to the one or more neuromodulators is about 500:1.

In some embodiments, the counter ion polymer is selected from the group consisting of dextran sulfate (DS), heparin (heparin sulfate), hyaluronic acid, and combinations thereof.

In some embodiments, the method further comprises adjusting the mixture of the one or more neuromodulators and the carrier molecule to a pH of about 3, including a pH of about 2.5, 2.6, 2.7, 2.8, 2.9, 3.0, 3.1, 3.2, 3.3, 3.4, and 3.5.

In some embodiments, the neuromodulator-encapsulated polyelectrolyte nanocomplexes (PNCs) have a Z-average particle size of about 20 nm to about 900 nm, including about 20, 30, 40, 50, 60, 70, 80, 90, 100, 200, 300, 400, 500, 600, 700, 800, and 900 nm, and with a size distribution (PDI) of about 0.1 to about 0.4, including about 0.1, 0.2, 0.3, and 0.4.

In other embodiments, the neuromodulator-encapsulated polyelectrolyte nanocomplexes (PNCs) have a Z-average particle size of about 60 nm, including about 50, 51, 52, 53, 54, 55, 56, 57, 58, 59, and 60, and with a size distribution (PDI) of about 0.1, including about 0.08, 0.09, 0.1, 0.11, and 0.12.

In some embodiments, the neuromodulator-encapsulated polyelectrolyte nanocomplexes (PNCs) have a negative surface charge with an average zeta potential of about −45 mV, including about −35, −36, −37, −38, −39, −40, −41, −42, −43, −44, −45, −46, −47, −48, −49, and −50 mV.

In some embodiments, the neuromodulator-encapsulated polyelectrolyte nanocomplexes (PNCs) have an encapsulation efficiency of about 80% to about 99%, including about 80, 81, 82, 83, 84, 85, 86, 87, 88, 89, 90, 91, 92, 93, 94, 95, 96, 97, 98, and 99% encapsulation efficiency.

In some embodiments, the method comprises a loading level of about 50%, including about 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, and 65% loading level.

In some embodiments, the neuromodulator-encapsulated polyelectrolyte nanocomplexes (PNCs) have a release rate of the neuromodulator of about 70%, including about 65, 66, 67, 68, 69, 70, 71, 72, 73, 74, and 75% release rate in about 24 hours; about 87%, including about 85, 86, 87, 88, and 89% release rate, in about 3 days; and about 90%, including about 90, 91, 92, 93, 94, and 95% release rate, in about 4 days.

In some embodiments, the neuromodulator-encapsulated polyelectrolyte nanocomplexes (PNCs) have a loading level of about 10% to about 70%, including 10, 15, 20, 25, 30, 35, 40, 45, 50, 55, 60, 65, and 70%.

In some embodiments, neuromodulator-encapsulated polyelectrolyte nanocomplexes (PNCs) has a release duration of about 1 to about 7 days, including about 1, 2, 3, 4, 5, 6, and 7 days.

### D. Microgels Comprising One or More Neuromodulators

In some embodiments, the presently disclosed subject matter provides a microgel or microgel particles comprising one or more neuromodulators.

Microgel particles serve the following roles: retain the complex at the injection site for an extended period of time, protect the complex from being endocytosed by macrophages or other tissue cells, improve the shelf stability, and facilitate lyophilization and reconstitution.

In other embodiments, the presently disclosed subject matter provides a microgel comprising a nanoparticle or a polyelectrolyte nanocomplex (PNC) comprising one or more neuromodulators, a carrier molecule, and a counter ion polymer, wherein the counter ion polymer has a charge enabling it to bind electrostatically to the one or more neuromodulators; and a crosslinked hydrophilic polymer, wherein the nanoparticle or polyelectrolyte nanocomplex (PNC) is distributed throughout the crosslinked hydrophilic polymer.

In certain embodiments, the crosslinked hydrophilic polymer comprises a hydrogel. In certain embodiments, the hydrogel comprises a natural or synthetic hydrophilic polymer selected from the group consisting of hyaluronic acid, chitosan, heparin, alginate, fibrin, polyvinyl alcohol, polyethylene glycol, sodium polyacrylate, an acrylate polymers, and copolymers thereof. In particular embodiments, the hydrogel comprises a crosslinked hyaluronic acid.

In certain embodiments, the microgel comprises a plurality of microgel particles having a spherical or asymmetrical shape. In particular embodiments, the plurality of microgel particles have a nominal size ranging from about 10 μm to about 1,000 μm. In yet more particular embodiments, the microgel or the plurality of microgel polymers has a shear storage modulus from about 10 Pa to about 10,000 Pa.

In certain embodiments, the microgel comprises a polyelectrolyte nanocomplex (PNC) having a nominal size ranging from about 20 nm to about 900 nm.

In other embodiments, the microgel further comprising a biodegradable polymer. In certain embodiments, the biodegradable polymer is selected from the group consisting of poly(L-lactic acid) (PLLA), polyglycolic acid (PGA), poly (D,L-lactic-co-glycolic acid) (PLGA), polycaprolactone (PCL), their PEGylated block copolymers, and combinations thereof. In particular embodiments, the biodegradable polymer is selected from the group consisting of polyethylene glycol (PEG)-b-PLLA, PEG-b-PLGA, PEG-b-PCL, and combinations thereof. In certain embodiments, the microgel comprises a nanoparticle having a nominal size ranging from about 20 nm to about 900 nm.

In other embodiments, one or more neuromodulators can be added to the hydrogel phase, as well, to provide a bolus dose at the time of injection. In such embodiments, the fraction of neuromodulator loaded in the microgel phase among the total dose in the injected formulation can be from about 0 to about 0.9, including 0, 0.1, 0.2, 0.3, 0.4, 0.5, 0.6, 0.7, 0.8, and 0.9. For example, in certain embodiments, 50% of the BoNTA can be loaded in the complex and the remaining 50% can be loaded in the microgel phase as a free form. It also is possible to include two or more nanoparticle formulations with different release profiles as a way to improve the therapeutic outcomes.

In other embodiments, the presently disclosed subject matter provides a process for generating a plurality of microgel particles, the process comprising:
(a) mixing a nanoparticle or polyelectrolyte nanocomplex (PNC) comprising one or more neuromodulators, a carrier molecule, and a counter ion polymer, and optionally a biodegradable polymer, with a hydrogel precursor;
(b) forming a hydrogel comprising the nanoparticle or polyelectrolyte nanocomplex (PNC) comprising one or more neuromodulators, a carrier molecule, and a counter ion polymer, and optionally a biodegradable polymer; and
(c) mechanically breaking the hydrogel into a plurality of microgel particles.

In certain embodiments, the plurality of microgel particles has a nominal size ranging from about 10 μm to 1,000 μm.

### E. Methods for Delivering One or More Neuromodulators to a Subject and Methods of Treatment Thereof

In other embodiments, the presently disclosed subject matter provides a method for delivering one or more neuromodulators to a subject, the method comprising:
administering a nanoparticle or microgel comprising a complex comprising a pharmaceutical agent and a counter ion polymer wherein the counter ion polymer has a charge enabling it to bind electrostatically to the pharmaceutical agent; and a matrix comprising the complex distributed throughout a biodegradable polymer. In some embodiments, the method prevents or treats a disease. In some embodiments, the method prevents or treats the disease compared to a reference subject not administered the nanoparticle or microgel.

In some embodiments, the method comprises administering the subject a presently disclosed nanoparticle or microgel to prevent or treat a disease.

Neuromodulators can be used for cosmetic and therapeutic uses.

In cosmetic applications, neuromodulators can be used for reducing facial wrinkles, in particular the uppermost third of the face, including the forehead, glabellar frown lines, and crow's feet. Neuromodulators also can be used to treat so-called “gummy smiles,” in which the neuromodulator is injected into the hyperactive muscles of upper lip, which causes a reduction in the upward movement of lip thus resulting in a smile with a less exposure of gingiva. To do so, the neuromodulator typically is injected in the three lip elevator muscles that converge on the lateral side of the ala of the nose; the levator labii superioris (LLS), the levator labii superioris alaeque nasi (LLSAN) muscle, and the zygomaticus minor (ZMi).

Therapeutic uses of neuromodulators include, but are not limited to treating:
focal dystonias, such as cervical dystonia (CD) to reduce the severity of abnormal head position and neck pain associated with CD in adults;
chronic sialorrhea, i.e., drooling, in adults;
muscle spasticity, i.e., disorders characterized by overactive muscle movement, including upper motor neuron syndrome, such as cerebral palsy, post-stroke spasticity, post-spinal cord injury spasticity, spasms of the head and neck, eyelid, vagina, limbs, jaw, and vocal cords, or clenching of muscles, including muscles of the esophagus, jaw, lower urinary tract and bladder, and anus, and refractory overactive bladder;
other muscle disorders, including strabismus, i.e., improper eye alignment, blepharospasm, hemifacial spasm, infantile esotropia, restricted ankle motion due to lower-limb spasticity associated with stroke in adults, and lower-limb spasticity in pediatric patients two years of age and older;
excessive sweating, including excessive underarm sweating of unknown cause;
migraine headache, including prophylactic treatment of chronic migraine headache. In such treatments, a neuromodulator is injected into the head and/or neck;
neuropathic pain;
chronic pain, such as osteoarthritis pain, see, e.g., U.S. Pat. No. 10,537,619, including modifying the progression of osteoarthritis, see, e.g., U.S. Pat. No. 10,149,893, treatment of arthritic joints to reduce pain and improve range of motion;
allergy symptoms;
depression, see, e.g., U.S. Pat. No. 8,940,308; and
premature ejaculation.

More particularly, in some embodiments, the disease or condition is selected from the group consisting of a cosmetic condition, blepharospasm, hemifacial spasms, spasmodic torticollis, spasticities, dystonias, migraine, low back pain, cervical spine disorders, strabismus, hyperhidrosis and hypersalivation. In some embodiments, the cosmetic condition is pronounced wrinkling.

In some embodiments, the method of treatment includes reducing facial lines or wrinkles of the skin or for removing facial asymmetries. In such embodiments, the composition is locally administered by subcutaneous or intramuscular injection of a non-lethal dose into, or in vicinity of, one or more facial muscles or muscles involved in the formation of the wrinkle of the skin or the asymmetry. U.S. Pat. No. 9,572,871 to Marx et al. for High frequency application of botulinum toxin therapy, issued Feb. 21, 2017.

In some embodiments, the composition is injected into the frown line, horizontal forehead line, crow's feet, nose perioral fold, mental ceases, popply chin, or platysmal bands. In some embodiments, the injected muscle is selected from the group consisting of corrugator supercillii, orbicularis oculi, procerus, venter frontalis of occipitofrontalis, orbital part of orbicularis oculi, nasalis, upper lip, orbicularis oris, lower lip, depressor angulis oris, mentalis and platysma, which muscles are involved in forming such lines. U.S. Pat. No. 8,557,255 to Marx et al. for High frequency application of botulinum toxin therapy, issued Oct. 15, 2013.

In other embodiments, botulinum toxins can be used to treat a variety of headache-related disorders, including: migraine, U.S. Pat. No. 5,714,468, issued Feb. 3, 1998; headache, U.S. Patent Application Publication No. 2005019132, Ser. No. 11/039,506, filed Jan. 18, 2005; medication overuse headache, U.S. Patent Application Publication No. 20050191320, Ser. No. 10/789,180, filed Feb. 26, 2004; neuropsychiatric disorders, U.S. Pat. No. 7,811,587, issued Oct. 12, 2010; each of which is incorporated by reference in their entirely.

In some embodiments, botulinum toxins can be used to prophylactically treat, reduce the occurrence of or alleviating a headache in a subject suffering from chronic migraine headaches. In some embodiments, the method comprises local administration of a clostridial neurotoxin, such as a botulinum neurotoxin, to the frontalis, corrugator, procerus, occipitalis, temporalis, trapezius and cervical paraspinal muscles of the subject. The injection(s) can be to a defined tissue depth, made with a particular injection angle, wherein the frequency and number of the units of botulinum neurotoxin administered to each site of injection varies. See e.g., U.S. Pat. No. 10,729,751, to Blumenfeld et al., for “Injection paradigm for administration of botulinum toxins,” issued Aug. 4, 2020 (providing an injection protocol for treating headaches). For example, in one embodiments, about twenty units divided among four sites of injection of the frontalis; about ten units divided among two sites of injection to the corrugator; about five units to one site of injection to the procerus; about thirty units divided among six sites of injection to about forty units divided among eight sites of injection to the occipitalis; about forty units divided among eight sites of injection up to fifty units divided among ten sites of injection to the temporalis; about thirty units divided among six sites of injection up to about fifty units divided among ten sites of injection to the trapezius; and about twenty units divided among four sites of injection to the cervical paraspinal muscles.

Embodiments of the present disclosure provide a targeted, fixed injection paradigm directed to a specific set of muscles with a specific minimum number and volume of injections, and further provides for the additional/optional administration of additional botulinum toxin to specific site of selected muscles. In one embodiment, the fixed dosage (that is, a minimum dosage amount in accordance with the fixed amounts and locations specified in a package insert or prescribing information) of botulinum toxin is administered to the frontalis, corrugator, procerus, occipitalis, temporalis, trapezius and cervical paraspinal muscles of a patient, and further a variable amount of additional botulinum toxin can be added to four or less of the seven head/neck areas such that the total amount of botulinum toxin administered does not exceed a maximum total dosage as indicated in the package insert or prescribing information accompanying a botulinum toxin-containing medicament.

In some embodiments, the method comprises treating medication overuse headache disorder, including triptan overuse disorder, opioid overuse disorder, and combinations thereof. In some embodiments, the total amount of botulinum neurotoxin administered is from about 155 units to about 195 units of onabotulinumtoxinA. In some embodiments, the administration is by injection, including subcutaneous injection and intramuscular injection. See, e.g., U.S. Pat. No. 10,406,213 to Turkel et al., for Injection paradigm for administration of botulinum toxins, issued Sep. 10, 2019.

In some embodiments, the method comprises treating an externally-caused migraine headache. In some embodiments, the externally-caused chronic migraine headache is related to post-traumatic stress disorder (PTSD) or traumatic brain injury (TBI). See, e.g., U.S. Pat. No. 8,883,143 to Binder, for Treatment of traumatic-induced migraine headache, issued Nov. 11, 2014, which is incorporated herein by reference in its entirety; see also U.S. Pat. No. 8,420,106 to Binder for Extramuscular treatment of traumatic-induced migraine headache, issued Apr. 16, 2013, which is incorporated herein by reference in its entirety.

In some embodiments, the method comprises treating migraine associated vertigo. See, e.g., U.S. Pat. No. 8,722,060 to Binder for Method of treating vertigo, issued May 13, 2014, which is incorporated herein by reference in its entirety.

In some embodiments, the method comprises treating a migraine headache by extramuscular injection of the neurotoxin to unmyelinated C fibers at emerging nerve exit points, wherein said nerve exit points are one or more of the Great auricular, Auriculotemporal, Supraorbital, Supratrochlear, Infratrochlear, Infraorbital or Mental nerve exit points. See, e.g., U.S. Pat. No. 8,617,569 to Binder for Treatment of migraine headache with diffusion of toxin in non-muscle related foraminal sites, issued Dec. 31, 2013, which is incorporated by reference in its entirety. In other embodiments, the method comprises extramuscular injection into one or more of the frontal, parietal and occipital aponeurotic fascia in the scalp. See, e.g., U.S. Pat. No. 8,491,917 to Binder for Treatment of migraine headache with diffusion of toxin in non-muscle related areas of the head, issued Jul. 23, 2013, which is incorporated by reference in its entirety.

In some embodiments, the method minimizes adverse effects associated with clostridial toxin administration. In some embodiments, the adverse effects include ptosis, neck pain/weakness, headache, and combinations thereof. In some embodiments, a particular administration protocol or dosing regimen can be used to prevent or minimize adverse effects associated with the administration of a clostridial toxin, such as a botulinum toxin, for treating or alleviating a headache in a patient with chronic migraine, the method comprises locating one or more administration target, isolating the one or more administration target, administering a therapeutically effective amount of a clostridial toxin to the isolated one or more administration target; wherein the administrating step is by injection and wherein the administering step comprises limiting the injection to a defined tissue depth and injection angle. In some embodiments, the adverse effects comprise ptosis, neck pain and/or weakness, headache, or combinations thereof.

In some embodiments, the presently disclosed methods include treating diseases or conditions caused by or associate with hyperactive cholinergic innervation of muscles, including severe movement disorder or severe spasticity (e.g., by administering a total dosage of from about 500 U to about 2000 U of the neurotoxic component). See, e.g., U.S. Pat. No. 10,792,344 to Marx et al. for High frequency application of botulinum toxin therapy, issued Oct. 6, 2020, which is incorporated herein by reference in its entirety.

The term “hyperactive cholinergic innervation”, as used herein, relates to a synapse, which is characterized by an unusually high amount of acetylcholine release into the synaptic cleft. In some embodiments, the disease or condition is or involves dystonia of a muscle. In some embodiments, the dystonia is selected from the group consisting of cranial dystonia, blepharospasm, oromandibular dystonia of the jaw opening or jaw closing type, bruxism, Meige syndrome, lingual dystonia, apraxia of eyelid opening, cervical dystonia, antecollis, retrocollis, laterocollis, torticollis, pharyngeal dystonia, laryngeal dystonia, spasmodic dysphonia of the adductor type, spasmodic dysphonia of the abductor type, spasmodic dyspnea, limb dystonia, arm dystonia, task specific dystonias, writer's cramp, musician's cramps, golfer's cramp, leg dystonia involving thigh adduction, thigh abduction, knee flexion, knee extension, ankle flexion, ankle extension, equinovarus deformity, foot dystonia involving striatal toe, toe flexion, toe extension, axial dystonia, Pisa syndrome, belly dancer dystonia, segmental dystonia, hemidystonia, generalised dystonia, dystonia in Lubag, dystonia in corticobasal degeneration, tardive dystonia, dystonia in spinocerebellar ataxia, dystonia in Parkinson's disease, dystonia in Huntington's disease, dystonia in Hallervorden Spatz disease, dopa-induced dyskinesias/dopa-induced dystonia, tardive dyskinesias/tardive dystonia, paroxysmal dyskinesias/dystonias, kinesiogenic, non-kinesiogenic, and action-induced. In some embodiments, the dystonia involves a clinical pattern selected from the group consisting of torticollis, laterocollis, retrocollis, anterocollis, flexed elbow, pronated forearm, flexed wrist, thumb-in-palm and clenched fist.

In some embodiments, the affected muscle is selected from the group consisting of ipsilateral splenius, contralateral sternocleidomastoid, ipsilateral sternocleidomastoid, splenius capitis, scalene complex, levator scapulae, postvertebralis, ipsilateral trapezius, levator scapulae, bilateral splenius capitis, upper trapezius, deep postvertebralis, bilateral sternocleidomastoid, scalene complex, submental complex, brachioradialis, biceps brachialis, pronator quadratus pronator teres, flexor carpi radialis, flexor carpi ulnaris, flexor pollicis longus, adductor pollicis, flexor pollicis brevis/opponens, flexor digitorum superficialis, and flexor digitorum profundus.

In some embodiments, the disease or condition is or involves spasticity of a muscle. In some embodiments, the spasticity is or is associated with a spastic condition in encephalitis and myelitis relating to autoimmune processes, multiple sclerosis, transverse myelitis, Devic syndrome, viral infections, bacterial infections, parasitic infections, fungal infections, hereditary spastic paraparesis, postapoplectic syndrome resulting from hemispheric infarction, postapoplectic syndrome resulting from brainstem infarction, postapoplectic syndrome resulting from myelon infarction, a central nervous system trauma, a central nervous system hemorrhage, an intracerebral hemorrhage, a subarachnoidal hemorrhage, a subdural hemorrhage, an intraspinal hemorrhage, a neoplasia, post-stroke spasticity, and spasticity caused by cerebral palsy. In some embodiments, the muscle is a smooth or striated muscle.

In some embodiments, the disease or condition is related to hyperactive exocrine glands. In some embodiments, the hyperactive exocrine gland is selected from the group consisting of sweat glands, tear glands, salivary glands and mucosal glands. U.S. Pat. No. 9,572,871 to Marx et al. for High frequency application of botulinum toxin therapy, issued Feb. 21, 2017; U.S. Pat. No. 9,095,523 to Marx et al. for High frequency application of botulinum toxin therapy, issued Aug. 4, 2015.

In some embodiments, the method comprises a method for decreasing depression in a patient by local administration of a botulinum neurotoxin to the frontalis, corrugator, procereus, occipitalis, temporalis, trapezius and cervical paraspinal muscles. See, e.g., U.S. Pat. No. 8,940,308 to Turkel et al. for Methods for treating depression, issued Jan. 27, 2015, which is incorporated by reference in its entirety.

In some embodiments, the disease or condition comprises nociceptive pain. As used herein, the term “nociceptive pain” is defined as pain that arises from actual or potential damage to non-neuronal tissue and is due to the physiological activation of nociceptors. As used herein, the term “neuropathic pain” is defined as pain arising as a direct consequence of a lesion or disease of the somatosensory nerve system.

In some embodiments, the disease or condition comprises treating or alleviating osteoarthritis pain. See, e.g., U.S. Pat. No. 10,537,619 to Turkel et al., for Methods for treating osteoarthritis pain, issued Jan. 21, 2020, which is incorporate herein by reference in its entirety. In some embodiments, the method comprises locally administering a therapeutically effective amount of a clostridial derivative to an osteoarthritis-affected site of the subject. In some embodiments, the therapeutically effective amount is from about 200 units to about 800 units, including about 200, 250, 300, 350, 400, 450, 500, 550, 600, 650, 700, 750, and 800 units. In some embodiments, the osteoarthritis-affected site is selected from the group consisting of a knee joint, a hip joint, a hand joint, a shoulder joint, an ankle joint, a foot joint, an elbow joint, a wrist joint, a sacroiliac joint, a spine joint, and combinations thereof. In some embodiments, the administering is by intra-articular injection into a joint space.

In some embodiments, the method includes a method for modifying the levels and/or activities of at least one agent associated with osteoarthritis-mediated cartilage degradation. See, e.g., U.S. Pat. No. 10,149,893 to Jiang et al. Methods for modifying progression of osteoarthritis, issued Dec. 11, 2018, which is incorporated herein by reference in its entirety. In such embodiments, the therapeutically effective amount can be from about 300 units to about 500 units. In some embodiments, the at least one agent associated with osteoarthritis-mediated cartilage degradation comprises a cartilage-degrading agent, a cartilage-forming component, or mixtures thereof. In certain embodiments, the cartilage-degrading agent is a proteinase. In particular embodiments, the proteinase is selected from the group consisting of metalloproteinases, cysteine proteinases, aspartate proteinases, serine proteinases, and combinations thereof. In certain embodiments, the cartilage-forming component is selected from the group consisting of aggrecan, proteoglycans, collagens, hyaluronan, and combinations thereof. In some embodiments, the osteoarthritis-affected site is selected from the group consisting of a knee joint, a hip joint, a hand joint, a shoulder joint, an ankle joint, a foot joint, an elbow joint, a wrist joint, a sacroiliac joint, a spine joint, and combinations thereof. In some embodiments, the method further comprises alleviating osteoarthritis associated pain.

In other embodiments, the presently disclosed subject matter provides a method for using a presently disclosed sustained release formulation, the method comprising local administration by injection of a sustained release formulation, wherein the one or more neuromodulators is released from the sustained release formulation over a period of time between about 3 days to about 200 days, including about 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, and 200 days, thereby treating a disease or condition selected from the group consisting of a cosmetic condition, focal dystonias, cervical dystonia (CD), chronic sialorrhea, and muscle spasticity.

As used herein, the term, “i.m.” refers to administration via an intramuscular route in which the therapeutic agent is deposited directly into vascular muscle tissue.

As used herein, the term “intra-articular injection” refers to an injection directly into a joint or into a portal.

As used herein, the term “extra-articular injection” refers to an injection outside of a joint space.

As used herein, the term “peri-articular injection” refers to an injection to an area around a joint.

As used herein, the term “local administration” means administration of a clostridial derivative to or to the vicinity of an arthritis-affected site in a patient by a non-systemic route. Thus, local administration excludes systemic routes of administration, such as intravenous or oral administration.

As used herein, the term “peripheral administration” means administration to a location away from a symptomatic location, as opposed to a local administration.

As used herein, the terms “administration,” or “to administer” means the step of giving (i.e., administering) a botulinum toxin to a subject, or alternatively a subject receiving a pharmaceutical composition. The present method can be performed via administration routes including intramuscular, non-intramuscular, intra-articular, extra-articular, peri-articular, intradermal, subcutaneous administration, topical administration (using liquid, cream, gel or tablet formulation), intrathecal administration, intraperitoneal administration, intravenous infusion, implantation (for example, of a slow-release device such as polymeric implant or miniosmotic pump), or combinations thereof.

As used herein, the terms “treating” or “treatment” means to prevent, reduce the occurrence, alleviate, or to eliminate an undesirable condition, for example headache, either temporarily or permanently.

As used herein, the term “alleviating” means a reduction of an undesirable condition or its symptoms, for example headache intensity or headache-associated symptoms. Thus, alleviating includes some reduction, significant reduction, near total reduction, and total reduction. An alleviating effect may not appear clinically for between 1 to 7 days after administration of a clostridial derivative to a patient or sometime thereafter.

As used herein, the term “therapeutically effective amount” refers to an amount sufficient to achieve a desired therapeutic effect. The therapeutically effective amount usually refers to the amount administered per injection site per patient treatment session, unless indicated otherwise.

The therapeutically effective amount of the clostridial derivative, for example a botulinum toxin can vary according to the potency of the toxin and particular characteristics of the condition being treated, including its severity and other various patient variables including size, weight, age, and responsiveness to therapy.

The biological activity of a neurotoxin is commonly expressed in Mouse Units (MU). As used herein, 1 MU is the amount of neurotoxic component, which kills 50% of a specified mouse population, e.g., a group of 18 to 20 female Swiss-Webster mice, weighing about 20 grams each, after intraperitoneal injection, i.e., the mouse i.p. LD₅₀ (Schantz & Kauter, 1978). The terms “MU” and “Unit” or “U” are interchangeable. Alternatively, the biological activity may be expressed in Lethal Dose Units (LDU)/ng of protein (i.e., neurotoxic component). The term “MU” is used herein interchangeably with the terms “U” or “LDU.” Assays exist for determining the biological activity of a clostridial neurotoxin. See, for example, U.S. Pat. No. 9,310,386 to Wilk et al. for In vitro assay for quantifying clostridial neurotoxin activity, issued Apr. 12, 2016.

One of ordinary skill in the art would recognize that commercially available Botulinum toxin formulations do not have equivalent potency units. In an illustrative example, one unit of BOTOX® (onabotulinumtoxinA), a botulinum toxin type A available from Allergan, Inc., has a potency unit that is approximately equal to 3 to 5 units of DYSPORT® (abobotulinumtoxinA), also a botulinum toxin type A available from Ipsen Pharmaceuticals. In some embodiments, the amount of abobotulinumtoxinA, (such as DYSPORT®), administered in the present method is about three to four times the amount of onabotulinumtoxinA (such as BOTOX®) administered, as comparative studies have suggested that one unit of onabotulinumtoxinA has a potency that is approximately equal to three to four units of abobotulinumtoxinA. MYOBLOC®, a botulinum toxin type B available from Elan, has a much lower potency unit relative to BOTOX®.

In some embodiments, the botulinum neurotoxin can be a pure toxin, devoid of complexing proteins, such as XEOMIN® (incobotulinumtoxinA). One unit of incobotulinumtoxinA has potency approximately equivalent to one unit of onabotulinumtoxinA. The quantity of toxin administered, and the frequency of its administration will be at the discretion of the physician responsible for the treatment and will be commensurate with questions of safety and the effects produced by a particular toxin formulation.

To guide the practitioner, in some embodiments, for example for treatment of headaches, typically, no less than about 1 unit and no more than about 25 units of a botulinum toxin type A (such as BOTOX®) is administered per injection site per patient treatment session. For a botulinum toxin type A, such as DYSPORT®, no less than about 2 units and no more than about 125 units of the botulinum toxin type A are administered per injection site, per patient treatment session. For a botulinum toxin type B, such as MYOBLOC®, no less than about 40 units and no more than about 1500 units of the botulinum toxin type B are administered per injection site, per patient treatment session.

In some embodiments, for BOTOX® no less than about 2 units and no more about 20 units of a botulinum toxin type A are administered per injection site per patient treatment session; for DYSPORT® no less than about 4 units and no more than about 100 units are administered per injection site per patient treatment session; and; for MYOBLOC®, no less than about 80 units and no more than about 1000 units are administered per injection site, per patient treatment session.

In other embodiments, for BOTOX® no less than about 5 units and no more about 15 units of a botulinum toxin type A; for DYSPORT® no less than about 20 units and no more than about 75 units, and; for MYOBLOC®, no less than about 200 units and no more than about 750 units are, respectively, administered per injection site, per patient treatment session.

Generally, the total amount of botulinum toxin suitable for administration to a subject should not exceed about 300 units, about 1,500 units or about 15,000 units respectively, per treatment session, depending on the biological activity or potency of the particular botulinum toxin administered. More particularly, the botulinum toxin can be administered in an amount of between about 1 unit and about 3,000 units, or between about 2 units and about 2000 units, or between about 5 units and about 1000 units, or between about 10 units and about 500 units, or between about 15 units and about 250 units, or between about 20 units and about 150 units, or between 25 units and about 100 units, or between about 30 units and about 75 units, or between about 35 units and about 50 units, or the like.

In some embodiments, the presently disclosed subject matter provides a sustained-release profile for neuromodulator formulations having at least a 120-day maximally effective release period, and, in some embodiments, extending the total duration of effect to between about 6 months and about 9 months, including about 6 months, 7 months, 8 month, and 9 months. Thus, the presently disclosed formulation provides for the long-term release of a neuromodulator, with a release duration ranging from about 1 month to 9 months, including about 1 month, 2 months, 3 months, 4 months, 5 months, 6 months, 7 months, 8 months, and 9 months. In some embodiments, the release duration is about 5 months, e.g., about 150 days. In some embodiments, the release duration is between about 3 days to about 200 days, including about 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, and 200 days.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In certain embodiments, the method comprises administering two or more formulations of the nanoparticle or microgel, wherein the two or more formulations of the nanoparticle or microgel each have a different release profile.

In some embodiments, the presently disclosed method further comprises administering one or more additional therapeutic agents in combination with the presently disclosed nanoparticles.

The term “combination” is used in its broadest sense and means that a subject is administered at least two agents, more particularly the presently disclosed nanoparticles and at least one additional therapeutic agent. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, the nanoparticles described herein can be administered alone or in combination with adjuvants that enhance stability of the nanoparticle formulation, alone or in combination with one or more agents, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of the presently disclosed nanoparticles and at least one additional therapeutic agent can be varied so long as the beneficial effects of the combination of these agents are achieved. Accordingly, the phrase “in combination with” refers to the administration of the presently disclosed nanoparticles and at least one additional therapeutic agent either simultaneously, sequentially, or a combination thereof. Therefore, a subject administered a combination of the presently disclosed nanoparticles and at least one additional therapeutic agent can receive the presently disclosed nanoparticles and at least one additional therapeutic agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the presently disclosed nanoparticles and at least one additional therapeutic agent are administered simultaneously, they can be administered to the subject as separate pharmaceutical compositions, each comprising either the presently disclosed nanoparticles or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent.

### F. Formulations and Methods of Administration

In some embodiments, the presently disclosed subject matter provides a sustained release formulation comprising a presently disclosed nanoparticle, wherein the formulation provides an effective concentration of the one or more neuromodulators in soft tissue for a period of time between about 3 days to about 200 days, including about 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, and 200 days.

In particular embodiments, a subject may be given, or administered, a nanoparticle comprising one or more neuromodulators. The nanoparticles may be administered to a subject in solid, liquid or aerosol form. The nanoparticles can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

Further, the presently disclosed nanoparticles can be provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The presently disclosed nanoparticles can be combined with the carrier in any convenient and practical manner, i.e., by solution, suspension, emulsification, admixture, encapsulation, absorption and the like. Such procedures are routine for those skilled in the art.

In some embodiments, the presently disclosed nanoparticles can be combined or mixed thoroughly with a semi-solid or solid carrier. The mixing can be carried out in any convenient manner such as grinding. Stabilizing agents can be also added in the mixing process in to protect the composition from loss of therapeutic activity, i.e., denaturation in the stomach. Examples of stabilizers for use in the composition include buffers, amino acids such as glycine and lysine, carbohydrates such as dextrose, mannose, galactose, fructose, lactose, sucrose, maltose, sorbitol, mannitol, and the like.

In further embodiments, the presently disclosed subject matter includes the use of pharmaceutical lipid vehicle compositions that include the presently disclosed nanoparticles and one or more lipids, and an aqueous solvent. As used herein, the term “lipid” includes any of a broad range of substances that are characteristically insoluble in water and extractable with an organic solvent. Examples include compounds which contain long-chain aliphatic hydrocarbons and their derivatives. A lipid may be naturally occurring or synthetic (i.e., designed or produced by man). Naturally occurring lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Compounds other than those specifically described herein that are understood by one of skill in the art as lipids also are encompassed by the presently disclosed compositions and methods.

One of ordinary skill in the art would be familiar with the range of techniques that can be employed for dispersing one or more nanoparticles in a lipid vehicle. For example, the one or more nanoparticles of the present invention may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of the presently disclosed nanoparticles administered to a subject can be determined by physical and physiological factors, such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In some embodiments, the presently disclosed nanoparticles of the present invention may be administered via a parenteral route. As used herein, the term “parenteral” includes routes that bypass the alimentary tract. Specifically, the pharmaceutical compositions disclosed herein may be administered for example, but not limited to intradermally, intramuscularly, or subcutaneously.

The presently disclosed formulations may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

In some embodiments, isotonic agents, for example, sugars or sodium chloride are included. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

In some embodiments, the composition comprises a pH buffer. In some embodiments, the pH buffer is sodium acetate. In some embodiments, the composition comprises a cryoprotectant. In some embodiments, the cryoprotectant is a polyalcohol. In some embodiments, the polyalcohol is selected from one or more of mannitol, inositol, lactilol, isomalt, xylitol, erythritol, sorbitol, and mixtures thereof. In some embodiments, the composition comprises a sugar. In some embodiments, the sugar is selected from monosaccharides, disaccharides, polysaccharides, and mixtures thereof. See, for example, U.S. Pat. No. 10,105,421 to Taylor for Therapeutic composition with a botulinum neurotoxin, issued, Oct. 23, 2018.

In some embodiments, the formulation comprises a detergent. The term “detergent” as used herein relates to any substance employed to solubilize or stabilize another substance, which may be either a pharmaceutical active ingredient or another excipient in a formulation. The detergent may stabilize said protein or peptide either sterically or electrostatically. The term “detergent” is used synonymously with the terms “surfactants” or “surface active agents”.

In some embodiments, the detergent is selected from the group consisting of non-ionic surfactants. The term “non-ionic surfactants” refers to surfactants having no positive or negative charge. In some embodiments, the non-ionic surfactants are selected from the group consisting of sorbitan esters (sorbitan monolaurate, sorbitan monopalmitate, sorbitan monostearate, sorbitan tristearate, sorbitan monooleate, Sorbitan trioleate), polysorbates (polyoxyethylene (20) sorbitan monolaurate (Polysorbate 20), polyoxyethylene (20) sorbitan monopalmitate, polyoxyethylene (20) Sorbitan monostearate, polyoxyethylene (20) sorbitan tristearate, polyoxyethylene (20) Sorbitan trioleate, Polyoxyethylene (20)-sorbitan-monooleate (Tween 80/Polysorbate 80)), poloxamers (poloxamer 407, poloxamer 188), cremophor, and mixture thereof.

In some embodiments, the detergent is anionic surfactant. The term “anionic surfactant” refers to surfactants comprising an anionic hydrophilic group. In some embodiments, the anionic surfactant is selected from the group consisting of tetradecyltrimethylammonium bromide, dodecyltrimethylammonium bromide, sodium laureth sulphate, sodium dodecyl sulphate (SDS), cetrimide, hexadecyltrimethylammonium bromide, and a mixture thereof.

In some embodiments, the detergent is a cationic surfactant. The term “cationic surfactant” encompasses surfactants comprising a cationic hydrophilic group. In some embodiments, the cationic surfactant is selected from the group consisting of benzalkonium chloride, cetyl trimethlammonium bromide (CTAB), cetylpyridinium chloride (CPC), benzethonium chloride (BZT), and mixtures thereof. See, for example, U.S. Pat. No. 9,198,856 to Burger et al. for Formulation for stabilizing proteins, which is free of mammalian excipient, issued Dec. 1, 2015; U.S. Pat. No. 9,173,944 to Taylor et al. for Formulation suitable for stabilizing proteins, which is free of mammalian excipients, issued Nov. 3, 2015.

### G. Kits

In some embodiments, the presently disclosed subject matter include a kit comprising the presently disclosed compositions. In a non-limiting example, the kit can comprise a presently disclosed nanoparticle (for example, a nanoparticle comprising one or more neuromodulators). The kits may comprise suitably aliquoted nanoparticles and, in some embodiments, one or more additional agents. The component(s) of the kits may be packaged either in aqueous media or in lyophilized form. The container of the kits will generally include at least one vial, test tube, flask, bottle, syringe or other container, into which a component may be placed, and preferably, suitably aliquoted. Where there is more than one component in the kit, the kit also will generally contain a second, third or other additional container into which the additional components may be separately placed. In other embodiments, various combinations of components may be comprised in a vial. The kits of the present invention also will typically include a means for containing the one or more nanoparticles of the present invention and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. The one or more nanoparticles may be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit. In other embodiments, the components of the kit may be provided as dried powder(s). When reagents and/or components are provided as a dry powder, the powder can be reconstituted by the addition of a suitable solvent. It is envisioned that the solvent may also be provided in another container means.

In some embodiments, the kit comprise prefilled glass or plastic syringes comprising the presently disclosed nanoparticles. See, for example, U.S. Pat. No. 10,549,042 to Vogt for Botulinum toxin prefilled glass syringe, issued Feb. 4, 2020, and U.S. Pat. No. 10,406,290 to Vogt for Botulinum toxin prefilled plastic syringe, issued Sep. 10, 2019, each of which are incorporated herein by reference in its entirety.

In other embodiments, a medical injection assembly for injecting onabotulinumtoxin A at plural injection sites in a patient's bladder wall to alleviate an overactive bladder condition is disclosed in U.S. Pat. No. 10,286,159 to Snoke et al., for Medical injection assemblies for onabotulinumtoxin A delivery and methods of use thereof, issued May 14, 2019, which is incorporated by reference in its entirety.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Representative Embodiments for Botulinum Toxin A (BoNTA) and BoNTA Toxoid

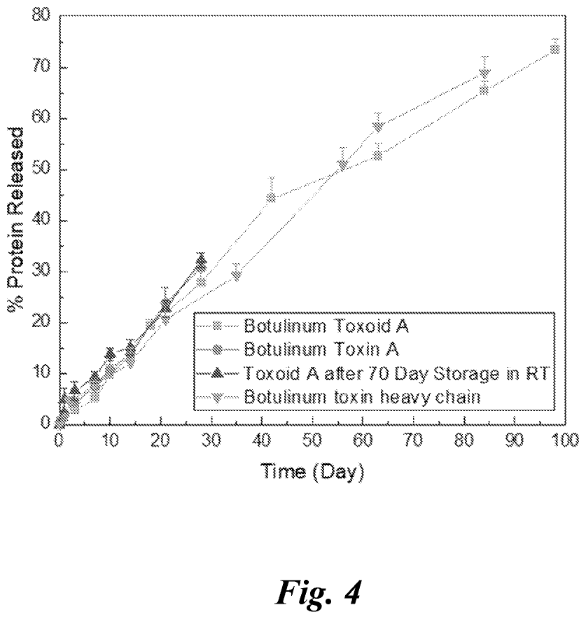

Experiments were conducted using both botulinum toxin A (BoNTA) and BoNTA toxoid (i.e., a partially deactivated form of BoNTA) to test the release profiles and reproducibility. Based on the composition of the NanoTox formulation, i.e., content or weight percent of BoNTA, filler protein, polyanion, and PLGA polymer, the release duration of BoNTA can be modulated from tens of hours to tens of weeks. For example, one specific formulation of NanoTox with BoNTA or BoNTA toxoid showed similar sustained release kinetics with approximately 65% protein release over an 84-day period with a near-linear profile (FIG. 4), projecting a total of more than 3.5 months of release duration. This release duration is an 8× extension of the release period alone compared to the current market leader Botox, which can only achieve a 14-day release duration. It is worth noting that the NanoTox formulation stored at room temperature for 70 days did not show significant different release kinetics (FIG. 4).

Given the high potency and extremely low EC50 for BoNTA and the need for re-synthesis and axonal transport of its target, the duration of effect can be extrapolated to more than 6 to 9 months.

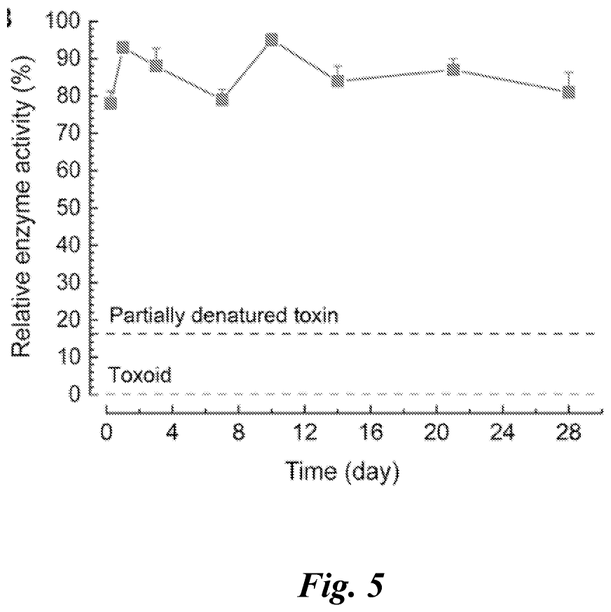

More importantly, a high level of bioactivity retention for BoNTA in this formulation has been demonstrated. The released BoNTA from the NanoTox system retaining a bioactivity of greater than 85% after 28 days at 37° C. as compared to the free form BoNTA (FIG. 5).

### Example 2

### Preparation of the Neuromodulator-Encapsulated Nanoparticles

The presently disclosed subject matter describes, in part, the preparation of nanoparticle formulations using BOTOX® (Botulinum type A toxin), or BoNTA toxoid, or BoNTA subunit (heavy chain). BoNTA, or BoNTA toxoid, or BoNTA heavy chain was dissolved in deionized (DI) water at a concentration of 2 mg/mL. The filler protein human serum albumin (HSA) or mouse serum albumin (MSA) was dissolved in DI water at a concentration of 2 mg/mL. The botox solution was mixed with the HSA solution at a protein weight ratio of 1:500, followed by adjusting the pH to 3.0 by adding 0.1 M HCl solution. One milliliter of this protein solution was then rapidly mixed with an equal volume of sodium dextran sulfate solution (DS, 2 mg/mL, pH was adjusted to 3.0) through the flash nanocomplexation (FNC) process using a confined impingement jet (CIJ) mixer with two inlets at a flow rate ranged 0.5 mL/min to 20 mL/min for both inlets. The outlet of the CIJ mixer was connected to another CIJ mixer with three inlets. The other two inlets of the mixer were streamed with 10 mg/mL PEG_{5K}-b-PLGA_{20K} (50:50) in acetonitrile and DI water separately, both at a flow rate of 2 mL/min. The protein encapsulated nanoparticles were obtained (BoNTA, NP1; BoNTA toxoid, NP2; BoNTA heavy chain, NP3).

The nanoparticles were dialyzed against DI water using dialysis membrane with molecular weight cut-off (MWCO) 3.5 KDa for 12 hours to remove acetonitrile with water being changed every 2 hours. The obtained solutions were purified by ultra-filtration using a filter with MWCO 100 KDa at 4,500 rpm for 20 min to remove the excess protein and DS.

The amount of unencapsulated protein was measured by the BCA assay, and the encapsulation efficiency (EE) was calculated using the following formula:

EE (%)=(mₜₒₜₐₗ−m_{free})/mₜₒₜₐₗ×100%,

where mₜₒₜₐₗ represents the mass of the total feeding protein and m_{free} represents the mass of free protein in the supernatant.

### Example 3

### Characterization of the Nanoparticles

The nanoparticles were characterized by particle size and zeta potential using a dynamic light scattering (DLS) Zetasizer Nano (Malvern Instruments, Worcestershire, UK). Each sample was measured for three runs and the data was reported as the mean±standard deviation of three readings.

Samples for TEM imaging were prepared by adding 10 microliters of nanoparticle solution onto an ionized copper grid covered with a carbon film. After 10 min, the solution was pipetted away, and a 6-microliter drop of 2% uranyl acetate was added to the grid. After 30 seconds, the solution was removed, and the grid was left to dry at room temperature. The samples were then imaged using a Technai FEI-12 electron microscope.

TABLE 1
Summary of particle size, PDI, zeta potential, EE, and loading level of
BoNTA proteins in nanoparticles

| | | | | | Encap- | |
|---|---|---|---|---|---|---|
| | | | | | sulation | |
| Nano- | | | | | effici- | |
| part- | | Average | | Zeta | ency | Loading |
| icle | Payload | size (nm) | PDI | potential | (%) | level (%) |
| NP1 | BoNTA | 96.8 ± 6.3 | 0.21 ± 0.01 | −24.8 ± 4.2 | 87.4 ± 2.9 | 13.9 ± 0.8 |
| NP2 | BoNTA | 86.4 ± 7.2 | 0.17 ± 0.02 | −27.2 ± 3.8 | 88.1 ± 3.6 | 14.2 ± 0.6 |
| | toxoid | | | | | |
| NP3 | BoNTA | 101.3 ± 8.5 | 0.23 ± 0.02 | −30.3 ± 5.1 | 83.2 ± 3.5 | 13.4 ± 0.6 |
| | Heavy | | | | | |
| | chain | | | | | |

The BoNTA-encapsulated PLGA nanoparticles (NP1 to NP3) were prepared with three different botox analogues at the same protein to polymer ratios and the same flow rates, showing a Z-average particle size ranging from 86 nm to 103 nm with a narrow size distribution (PDI values˜0.17-0.23) (Table 1). All the nanoparticles showed negative surface charges with zeta potential ranging from −25 to −30 mV. The encapsulation efficiencies ranged from 83% to 88%, while the loading levels ranged from 13.4% to 14.2%.

### Example 4

### Release Experiments for NP1-3 and Data Analysis

In vitro release of BoNTA/toxoid/heavy chain was conducted by 500 microliters of protein-loaded nanoparticle suspension containing 0.5 mg protein (BoNTA+HSA) mixed with the same volume of 2×PBS into a 1.5 mL Eppendorf centrifuge tube. The centrifuge tube was put into an incubator at 37° C. with an agitation rate of 100 rpm. Multiple tubes were prepared at the same method. At each designated time point, three tubes were obtained from the incubator and then were ultracentrifuged at 50,000 rcf for 30 min. The supernatant was collected and concentrated by lyophilization and further reconstituted using 100 microliters of DI water. An ELISA assay was employed to quantify the amount of released toxin.

NP1-3 all showed sustained release with 30-35% released within 30 days (FIG. 4). The toxin release duration can be extrapolated to 118 days and the toxoid release duration can be extrapolated to 147 days. NP2 that was stored in room temperature for 70 days also repeated the trend. NP3 with heavy chain encapsulated can be extrapolated to 110 days if 100% release has been assumed.

### Example 5

### Bioactivity of the Released BoNTA from NP1-3

Bioactivity of the released toxin was conducted by a fluorogenic SNAPtide cleavage assay. The released toxin was lyophilized and reconstituted with the reduction buffer (20 mM HEPES, pH 8.0, 5 mM DTT, 0.3 mM ZnSO₄ and 0.1% Tween 20). The concentration of toxin was normalized to the same as the standard sample of toxin. After 30 minutes incubation at 37° C., 100 μL of the solution was added into the 96-well plate with 150 μL of the reaction buffer (20 mM HEPES, pH 8.0, 1.25 mM DTT, 0.75 mM ZnSO₄ and 0.1% Tween 20). After incubation overnight at 37° C., the 96-well plate was then analyzed by a fluorometer at the excitation wavelength 320 nm and emission wavelength 420 nm. The bioactivity of released toxin was preserved with no significant change for 28 days with the toxoid has no bioactivity (FIG. 5).

### Example 6

### Preparation of the Neuromodulator-Encapsulated Polyelectrolyte Nanocomplex (PNC, NP4)

The presently disclosed subject matter describes, in part, the preparation of polyelectrolyte nanocomplex (PNC) formulations using BOTOX® (Botulinum type A toxin), or BoNTA toxoid, or BoNTA subunit (heavy chain). Using the process described in the example procedure below, one polyelectrolyte nanocomplex (PNC) formulation (NP4) with Botulinum type A toxin was prepared.

BoNTA was dissolved in deionized (DI) water at a concentration of 2 mg/mL. The filler protein human serum albumin (HSA) or mouse serum albumin (MSA) was dissolved in DI water at a concentration of 2 mg/mL. The BoNTA solution was mixed with the HSA solution at a protein weight ratio of 1:500, followed by adjusting the pH to 3.0 by adding 0.1 M HCl solution. One milliliter of this protein solution was then rapidly mixed with an equal volume of sodium dextran sulfate solution (DS, 2 mg/mL, pH was adjusted to 3.0) through the flash nanocomplexation (FNC) process using a confined impingement jet (CIJ) mixer with two inlets at a flow rate of 10 mL/min (range: 0.5 to 20 mL/min) for both inlets. The BoNTA/HSA/DS polyelectrolyte nanocomplexes (PNCs) (NP4) were collected and purified by dialysis against DI water at 4° C. Alternatively, the obtained polyelectrolyte nanocomplex (PNC) suspension was purified by ultra-filtration using a filter with MWCO 100 KDa at 4,500 rpm for 20 min to remove the excess protein and DS. The obtained polyelectrolyte nanocomplex (PNC) formulation is referred to as NP4.

### Example 7

### Characterization of the Polyelectrolyte Nanocomplex (PNC) Formulation NP4

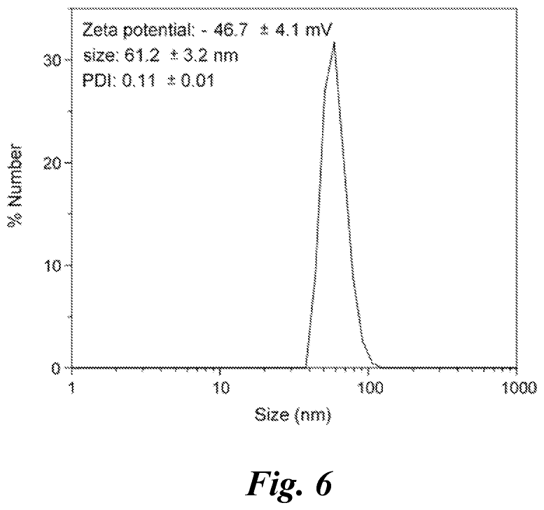

The polyelectrolyte nanocomplexes (PNCs) in NP4 were characterized by particle size and zeta potential by dynamic light scattering (DLS) using a Zetasizer Nano (Malvern Instruments). Each sample was measured for three runs and the data was reported as the mean±standard deviation of three readings. The BoNTA-encapsulated polyelectrolyte nanocomplexes (PNCs) showed a Z-average particle size of 61.2 nm with a narrow size distribution (PDI=0.11). The polyelectrolyte nanocomplexes (PNCs) showed negative surface charges with an average zeta potential at −46.7 mV (FIG. 6).

The amount of unencapsulated protein in NP4 was measured by the BCA assay, and the encapsulation efficiency (EE) was calculated using the following formula:

EE (%)=(mₜₒₜₐₗ−m_{free})/mₜₒₜₐₗ×100%,

where mₜₒₜₐₗ represents the mass of the total feeding protein and m_{free} represents the mass of free protein in the supernatant. The encapsulation efficiency was 98%, and the loading level was 49%.

### Example 8

### Release Experiments for NP4 and Data Analysis

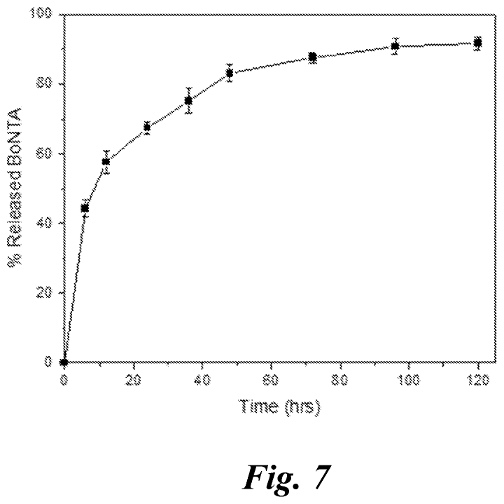

Experiments were conducted using botulinum toxin A (BoNTA) to test the release profiles and reproducibility. In vitro release of BoNTA from NP4 was conducted by 500 microliters of protein-loaded nanoparticle suspension containing 0.5 mg protein (BoNTA and HSA) mixed with the same volume of 2×PBS into a 1.5 mL Eppendorf centrifuge tube. Multiple samples in the tubes were agitated at 100 rpm under 37° C. in a shaker incubator. At each designated time point, three tubes were obtained from the incubator and then were ultracentrifuged at 50,000 rcf for 30 min. The supernatant was collected and concentrated by lyophilization and further reconstituted using 100 microliters of DI water. An ELISA assay was employed to quantify the amount of released toxin. A relatively fast release rate of BoNTA was observed from NP4, with approximately 70% BoNTA in 24 hours, 85% of BoNTA released in 3 days, and 91% of BoNTA released in 4 days (FIG. 7).

### Example 9

### Microgel Particle Formulation 1 (MP1) Loaded With NP4

### 9.1 Preparation of the Microgel Particle Formulation 1 (MP1)

Polyelectrolyte nanocomplexes (PNCs, NP4) was dispersed in 5 mg/mL (possible range: 1-40 mg/mL, including 1, 2, 3, 4, 5, 6, 7, 8, 9, 10, 11, 12, 13, 14, 15, 16, 17, 18, 19, 20, 21, 22, 23, 24, 25 26, 27, 28, 29, 30, 31, 32, 33, 34, 35, 36, 37, 38, 39, and 40) acrylated hyaluronic acid (HA-Ac, acrylation degree 5-20%, including 5, 6, 7, 8, 9, 10, 11, 12, 13, 14, 15, 16, 17, 18, 19, and 20%) in PBS at the concentration of 0.4 mg/mL (possible range: 0.01-10 mg/mL, including 0.01, 0.05, 0.1, 0.2, 0.3, 0.4, 0.5, 0.6, 0.7, 0.8, 0.9, 1, 2, 3, 4, 5, 6, 7, 8, 9, and 10 mg/mL) of total protein (neuromodulator+HSA). A pre-determined amount of thiolated PEG (PEG-SH; concentration range: 4-12.8 mg/mL, including 4, 5, 6, 7, 8, 9, 10, 11, 12, and 12.8 mg/mL) was added to the suspension, and incubated overnight at 37° C. The crosslinked hydrogel was further processed into microgel particles (MPs) to improve the injectability. The microgel particles can be lyophilized with 9.5% (w/w) trehalose and stored in −20° C. freezer. This formulation is termed MP1.

9.2 Release Profile of BoNTA from the MP1

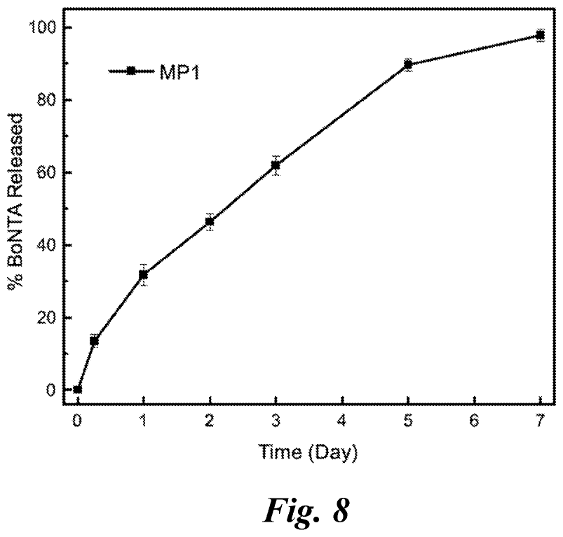

MP1 was reconstituted in a centrifuge tube that has been filled with 5 mL of PBS at 0.5 mg of total protein/mL. The MP suspension was incubated at 37° C. with 100 rpm agitation. At designated time point, the suspension was centrifuged at 4,500 rpm for 10 min to sediment MP′. An aliquot of supernatant (0.5 mL) was collected, and the same amount of fresh PBS was refilled. The centrifuge tube was then put back into the incubator. The collected supernatant was lyophilized and reconstituted with 100 mL DI water, followed by ELISA measurement. FIG. 8 show release profiles of BoNTA from MP1 (NP4 loaded in the HA hydrogel) incubated at 37° C. in PBS. As shown in FIG. 7, a relatively fast release rate of BoNTA was observed from NP4; whereas a slightly gradual release profile was observed when NP4 was loaded in MP1, with a total of BoNTA released out in 7 days (FIG. 8).

9.3 Bioactivity of the Released BoNTA from MP1

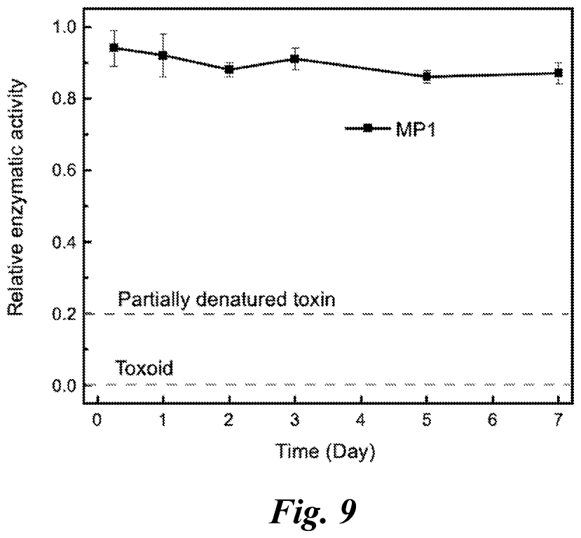

Bioactivity of the released BoNTA from the MP1 was conducted by a fluorogenic SNAPtide cleavage assay that was previously described in EXAMPLE 5. The release profile and bioactivity of released BoNTA was preserved with no significant change for 7 days, as shown in FIG. 9.

### Example 10

### Microgel Particle Formulation 2 (MP2) Loaded With NP1

### 10.1 Preparation of the Microgel Particle Formulation 2 (MP2)

NP1 was dispersed in 5 mg/mL (possible range: 1-40 mg/mL, including 1, 2, 3, 4, 5, 6, 7, 8, 9, 10, 11, 12, 13, 14, 15, 16, 17, 18, 19, 20, 21, 22, 23, 24, 25 26, 27, 28, 29, 30, 31, 32, 33, 34, 35, 36, 37, 38, 39, and 40) acrylated hyaluronic acid (HA-Ac, acrylation degree 5-20%, including 5, 6, 7, 8, 9, 10, 11, 12, 13, 14, 15, 16, 17, 18, 19, and 20%) in PBS at the concentration of 0.4 mg/mL (possible range: 0.01-10 mg/mL, including 0.01, 0.05, 0.1, 0.2, 0.3, 0.4, 0.5, 0.6, 0.7, 0.8, 0.9, 1, 2, 3, 4, 5, 6, 7, 8, 9, and 10 mg/mL) of total protein (neuromodulator+HSA). A pre-determined amount of thiolated PEG (PEG-SH; concentration range: 4-12.8 mg/mL, including 4, 5, 6, 7, 8, 9, 10, 11, 12, 12.5, and 12.8 mg/mL) was added to the suspension, and incubated overnight at 37° C. The crosslinked hydrogel was further processed into microgel particles to improve the injectability. The microgel particles can be lyophilized with 9.5% (w/w) trehalose and stored in −20° C. freezer. This formulation is termed MP2.

10.2 Release Profile of BoNTA from the MP2

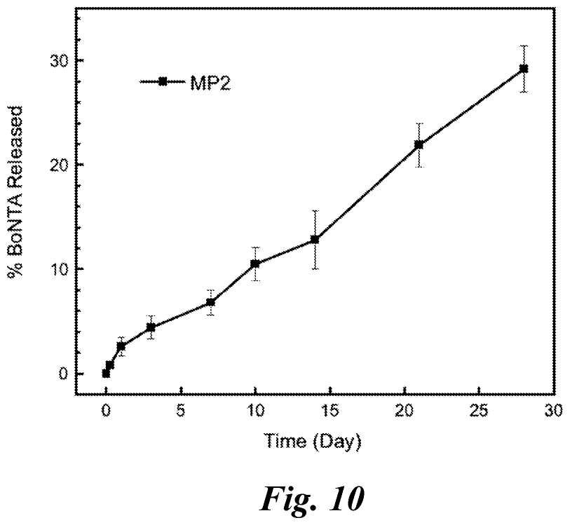

MP2 was reconstituted in a centrifuge tube that has been filled with 5 mL of PBS at 0.5 mg of total protein/mL. The MP suspension was incubated at 37° C. with 100 rpm agitation. At designated time point, the suspension was centrifuge at 4,500 rpm for 10 min to sediment MP2. An aliquot of supernatant (0.5 mL) was collected, and the same amount of fresh PBS was refilled. The centrifuge tube was then put back to the incubator. The collected supernatant was lyophilized and reconstituted with 100 μL DI water, followed by ELISA measurement. FIG. 10 shows the release profiles of BoNTA from MP2 incubated at 37° C. in PBS. A sustained release profile was maintained for BoNTA from this microgel particle formulation.

10.3 Bioactivity of the Released BoNTA from MP2

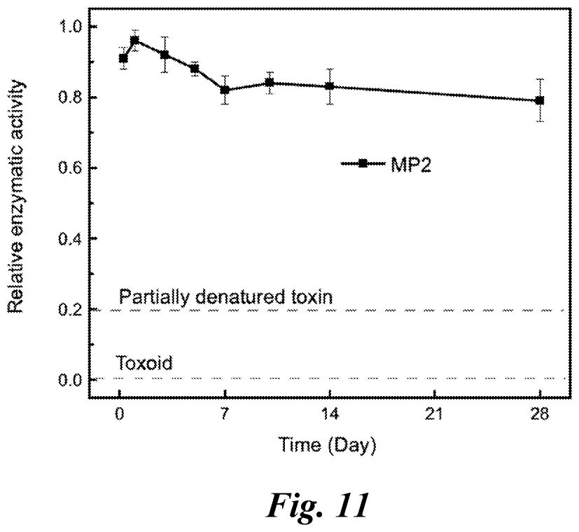

Bioactivity of the released BoNTA from the MP2 was conducted by a fluorogenic SNAPtide cleavage assay that was previously described in EXAMPLE 5. The release profile and bioactivity of released BoNTA was preserved with no significant change for 7 days as shown in FIG. 11.

### Example 11

### Microgel Particle Formulation 3 (MP3): NP1 Loaded in Nanofiber-Hydrogel Composite (NHC)

### 11.1 Preparation of the Microgel Particle Formulation 3 (MP3)

NP1 was dispersed in 5 mg/mL (possible range: 1-40 mg/mL, including 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40) acrylated hyaluronic acid (HA-Ac, acrylation degree 5-20%, including 5, 6, 7, 8, 9, 10, 11, 12, 13, 14, 15, 16, 17, 18, 19, and 20%) in PBS at the concentration of 0.4 mg/mL (possible range: 0.01-10 mg/mL, including 0.01, 0.05, 0.1, 0.2, 0.3, 0.4, 0.5, 0.6, 0.7, 0.8, 0.9, 1, 2, 3, 4, 5, 6, 7, 8, 9, and 10 mg/mL) of total protein (neuromodulator+HSA), and 10 mg/mL [range: 50 mg/mL, including 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50 mg/mL] electrospun polycaprolactone nanofiber fragments (fiber diameter in a range of 0.2 to 2 μm, including 0.2, 0.3, 0.4, 0.5, 0.6, 0.7, 0.8, 0.9, 1.0, 1.1, 1.2, 1.3, 1.4, 1.5, 1.6, 1.7, 1.8, and 1.9 and 2 μm; length in a range of 20 to 100 μm, including 20, 25, 30, 35, 40, 45, 50, 55, 60, 65, 70, 75, 80, 85, 90, 95, and 100 μm) suspended evenly in the solution. A pre-determined amount of thiolated PEG (PEG-SH; concentration range: 4-12.8 mg/mL, including 4, 5, 6, 7, 8, 9, 10, 11, 12, 12.5, and 12.8 mg/mL) was added to the suspension, and incubated overnight at 37° C. The crosslinked hydrogel was further processed into microgel particles to improve the injectability. The microgel particles can be lyophilized with 9.5% (w/w) trehalose and stored in −20° C. freezer. This formulation is referred to as MP3.

11.2 Release Profile of BoNTA from the MP3

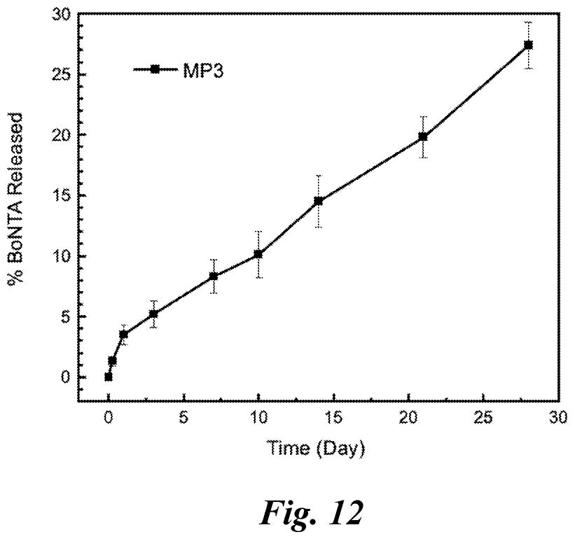

MP3 was reconstituted in a centrifuge tube that has been filled with 5 mL of PBS at 0.5 mg of total protein/mL. The MP suspension was incubated at 37° C. with 100 rpm agitation. At designated time point, the suspension was centrifuge at 4,500 rpm for 10 min to sediment MP3. An aliquot of supernatant (0.5 mL) was collected, and the same amount of fresh PBS was refilled. The centrifuge tube was then put back to the incubator. The collected supernatant was lyophilized and reconstituted with 100 mL DI water, followed by ELISA measurement. FIG. 12 shows the release profiles of BoNTA from MP2 incubated at 37° C. in PBS. A sustained release profile was achieved for BoNTA from this microgel particle formulation.

### Example 12

### Bioactivity and Therapeutic Function of the Released BoA in Target Muscle in Sprague-Dawley Rats

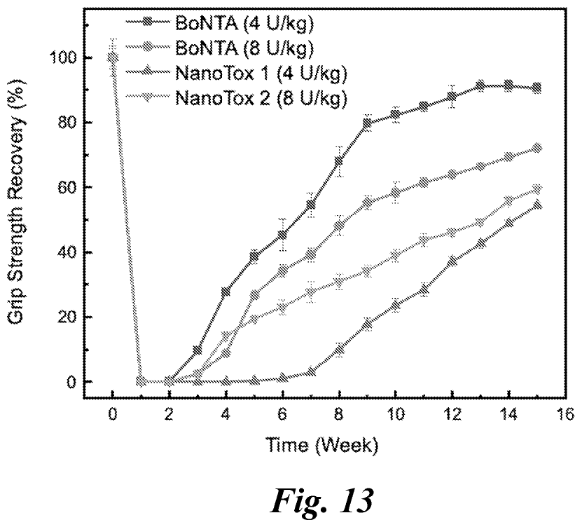

To test in vivo performance, the effect of NanoTox treatment on muscle relaxation after a single intramuscular injection into the forelimbs (flexor digitorum profundus, flexor digitorum superficials) in Sprague-Dawley rats was measured. Unencapsulated BoNTA injections (at 4 U/kg and 8 U/kg) were used as controls. At these doses tested, full paralysis was observed in all animals receiving injections. Rats were assessed weekly for stimulated grip strength of the forelimbs and the maximum force applied was recorded in triplicates and used to compare with the baseline that was measured before the injections and reported as percent of grip strength recovery. FIG. 13 compares the functional recovery rates of two NanoTox formulations (NanoTox 1 and NanoTox 2), and bolus BoNTA injections at 4 U/kg and 8 U/kg dose levels.

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A polyelectrolyte nanocomplex (PNC) comprising one or more neuromodulators, a carrier molecule, and a counter ion polymer, wherein the counter ion polymer has a charge enabling it to bind electrostatically to the one or more neuromodulators.

2. A nanoparticle comprising the polyelectrolyte nanocomplex (PNC) of claim 1 and a non-water-soluble biodegradable polymer, wherein the polyelectrolyte nanocomplex (PNC) is distributed throughout the non-water-soluble biodegradable polymer.

3. The PNC of claim 1 or the nanoparticle of claim 2, wherein the one or more neuromodulators comprise a therapeutically active derivative of Clostridial neurotoxin.

4. The PNC or nanoparticle of claim 3, wherein the Clostridial neurotoxin comprises a therapeutically active derivative of a botulinum toxin.

5. The PNC or nanoparticle of claim 4, wherein the botulinum toxin is selected from the group consisting of therapeutically active derivatives of botulinum toxin types A, B, C, including C₁, D, E, F and G, and subtypes and mixtures thereof.

6. The PNC or nanoparticle of claim 5, wherein the one or more neuromodulators is selected from the group consisting of onabotulinumtoxin A, abobotulinumtoxin A, incobotulinumtoxin A, prabotulinumtoxin A, rimabotulinumtoxin B, and combinations thereof.

7. The PNC or nanoparticle of any one of claims 1-6, wherein the carrier molecule comprises a polyelectrolyte selected from the group consisting of a cationic polymer, a protein, and a polysaccharide.

8. The PNC or nanoparticle of claim 7, wherein the protein is selected from the group consisting of IgG, collagen, gelatin, and serum albumin.

9. The PNC or nanoparticle of any one of claims 1-8, wherein a weight ratio of the carrier molecule to the one or more neuromodulators can vary from about 1:1 to about 2000:1.

10. The PNC or nanoparticle of claim 9, wherein the weight ratio of the carrier molecule to the one or more neuromodulators is about 500:1.

11. The PNC or nanoparticle of any one of claims 1-10, wherein the counter ion polymer is selected from the group consisting of dextran sulfate (DS), heparin (heparin sulfate), hyaluronic acid, and combinations thereof.

12. The PNC or nanoparticle of any one of claims 1-11, wherein the biodegradable polymer is a copolymer selected from the group consisting of poly(L-lactic acid) (PLLA), polyglycolic acid (PGA), poly (D,L-lactic-co-glycolic acid) (PLGA), polycaprolactone (PCL), their PEGylated block copolymers, and combinations thereof.

13. The PNC or nanoparticle of claim 12, wherein the biodegradable polymer is selected from the group consisting of polyethylene glycol (PEG)-b-PLLA, PEG-b-PLGA, PEG-b-PCL, and combinations thereof.

14. The PNC or nanoparticle of claim 13, wherein the nanoparticle comprises one of: onabotulinumtoxinA (BoNTA):carrier protein:dextran sulfate (DS):PEG-b-PLGA in a m:1:1:n ratio, whereas m=0.0005 to 1, and n=3 to 10; (BoNTA+carrier):DS:PEG-b-PLGA is 1:1:5; or BoNTA:carrier is 1:1 to 1:2000.

15. A microgel comprising:
a polyelectrolyte nanocomplex (PNC) of claim 1 or the nanoparticle of claim 2 comprising one or more neuromodulators, a carrier molecule, and a counter ion polymer, wherein the counter ion polymer has a charge enabling it to bind electrostatically to the one or more neuromodulators; and
a crosslinked hydrophilic polymer, wherein the polyelectrolyte nanocomplex (PNC) of claim 1 or nanoparticle of claim 2 is distributed throughout the crosslinked hydrophilic polymer.

16. The microgel of claim 15, wherein the microgel comprises the polyelectrolyte nanocomplex (PNC) of claim 1 or the nanoparticle of claim 2, wherein the microgel has a weight ratio of polyelectrolyte nanocomplex (PNC) to nanoparticle ranging from about 0 to about 1.

17. The microgel of claim 15, wherein the microgel comprises a composite of the crosslinked hydrophilic polymer and a nanofiber.

18. The microgel of claim 17, wherein the composite comprises a plurality of polycaprolactone fibers having a mean length of less than about 200 micrometers, which are covalently linked to the crosslinked hydrophilic polymer.

19. The microgel of any one of claims 15-17, wherein the crosslinked hydrophilic polymer comprises a hydrogel.

20. The microgel of claim 19, wherein the hydrogel comprises a natural or synthetic hydrophilic polymer selected from the group consisting of hyaluronic acid, chitosan, heparin, alginate, fibrin, polyvinyl alcohol, polyethylene glycol, sodium polyacrylate, an acrylate polymers, and copolymers thereof.

21. The microgel of claim 20, wherein the hydrogel comprises a crosslinked hyaluronic acid.

22. The microgel of any one of claim 15-21, wherein the microgel comprises a plurality of microgel particles having a spherical or asymmetrical shape.

23. The microgel of claim 22, wherein the plurality of microgel particles have a nominal size ranging from about 10 μm to about 1,000 μm.

24. The microgel of any one of claims 15-23, wherein the microgel or the plurality of microgel polymers has a shear storage modulus from about 10 Pa to about 10,000 Pa.

25. The microgel of any one of claims 15-24, wherein the microgel comprises a polyelectrolyte nanocomplex (PNC) having a nominal size ranging from about 20 nm to about 900 nm.

26. The microgel of any one of claims 15-24, wherein the PNC or the nanoparticle comprises a biodegradable polymer selected from the group consisting of poly(L-lactic acid) (PLLA), polyglycolic acid (PGA), poly (D,L-lactic-co-glycolic acid) (PLGA), polycaprolactone (PCL), their PEGylated block copolymers, and combinations thereof.

27. The microgel of claim 26, wherein the biodegradable polymer is selected from the group consisting of polyethylene glycol (PEG)-b-PLLA, PEG-b-PLGA, PEG-b-PCL, and combinations thereof.

28. The microgel of any one of claims 26-27, wherein the microgel comprises a nanoparticle having a nominal size ranging from about 20 nm to about 900 nm.

29. The microgel of any one of claims 15-28, wherein the crosslinked hydrophilic polymer further comprises one or more neuromodulators added directly thereto.

30. The microgel of claim 29, wherein the one or more neuromodulators added directly to the crosslinked hydrophilic polymer is a fraction of an amount of the one or more neuromodulators in the nanoparticle or polyelectrolyte nanocomplex (PNC).

31. The microgel of claim 30, wherein the fraction of the one or more neuromodulators added directly to the crosslinked hydrophilic polymer has a range from about 0 to about 1.

32. A process for preparing a neuromodulator-encapsulated polyelectrolyte nanocomplex (PNC), the method comprising:
(a) mixing the aqueous solution of one or more neuromodulators and the aqueous solution of the carrier molecule to form a protein solution; and
(b) mixing the protein solution with a counter ion polymer by a flash nanocomplexation (FNC) process to form a neuromodulator-encapsulated polyelectrolyte nanocomplex (PNC).

33. The process of claim 32, wherein the one or more neuromodulators comprise a therapeutically active derivative of Clostridial neurotoxin.

34. The process of claim 33, wherein the Clostridial neurotoxin comprises a therapeutically active derivative of a botulinum toxin.

35. The process of claim 34, wherein the botulinum toxin is selected from the group consisting of therapeutically active derivatives of botulinum toxin types A, B, C, including C₁, D, E, F and G, and subtypes and mixtures thereof.

36. The process of claim 35, wherein the one or more neuromodulators is selected from the group consisting of onabotulinumtoxin A, abobotulinumtoxin A, incobotulinumtoxin A, prabotulinumtoxin A, rimabotulinumtoxin B, and combinations thereof.

37. The process of any one of claims 32-36, wherein the carrier molecule comprises a polyelectrolyte selected from the group consisting of a cationic polymer, a protein, and a polysaccharide.

38. The process of claim 37, wherein the protein is selected from the group consisting of IgG, collagen, gelatin, and serum albumin.

39. The process of any one of claims 32-38, wherein a weight ratio of the carrier molecule to the one or more neuromodulators can vary from about 1:1 to about 2000:1.

40. The process of claim 39, wherein the weight ratio of the carrier molecule to the one or more neuromodulators is about 500:1.

41. The process of any one of claims 32-40, wherein the counter ion polymer is selected from the group consisting of dextran sulfate (DS), heparin (heparin sulfate), hyaluronic acid, and combinations thereof.

42. The process of any one of claims 32-41, wherein the neuromodulator-encapsulated polyelectrolyte nanocomplexes (PNCs) have a Z-average particle size of about 20 nm to about 900 nm, and with a size distribution (PDI) of about 0.1 to about 0.4.

43. The process of any one of claims 32-42, wherein the neuromodulator-encapsulated polyelectrolyte nanocomplexes (PNCs) have a negative surface charge with an average zeta potential of about −30 mV to about −50 mV.

44. The process of any one of claims 32-43, comprising an encapsulation efficiency of about 80% to about 99%.

45. The process of any one of claims 32-44, comprising a loading level of about 10% to about 70%.

46. The process of any one of claims 32-45, wherein the neuromodulator-encapsulated polyelectrolyte nanocomplexes (PNCs) have a release duration of about 1 day to about 7 days.

47. A process for generating a plurality of nanoparticles, the process comprising:
(a) forming a polyelectrolyte nanocomplex (PNC) by mixing a preformed solution of one or more neuromodulators and one or more carrier molecules and a counter ion polymer using a first continuous mixing process;
(b) co-precipitating the polyelectrolyte nanocomplex (PNC) with a non-water soluble biodegradable polymer using a second continuous mixing process; and
(c) forming a plurality of nanoparticles, wherein the polyelectrolyte nanocomplex (PNC) comprising the one or more neuromodulators, one or more carrier molecules, and counter ion polymer is distributed throughout the non-water-soluble biodegradable polymer.

48. The process of claim 47, wherein step (a) and step (b) proceed simultaneously.

49. The process of claim 47, wherein the first continuous mixing process comprises a flash nanocomplexation (FNC) process.

50. The process of claim 47, wherein the forming of the polyelectrolyte nanocomplex (PNC) is by electrostatic attraction between the one or more neuromodulators and the counter ion polymer.

51. The process of claim 47, wherein the mixing of the polyelectrolyte nanocomplex (PNC) and the non-water soluble biodegradable polymer is by solvent-induced flash nanoprecipitation (FNP).

52. The process of claim 47, wherein the forming of the plurality of nanoparticles occurs by the precipitation of the non-water-soluble biodegradable polymer together with the polyelectrolyte nanocomplex (PNC).

53. The process of any one of claims 47-52, wherein the plurality of nanoparticles have a Z-average particle size of about 20 nm to about 900 nm, and with a size distribution (PDI) of about 0.1 to about 0.4.

54. The process of any one of claims 47-53, wherein the plurality of nanoparticles have a negative surface charge with an average zeta potential of about −10 mV to about −35 mV.

55. The process of any one of claims 47-54, comprising an encapsulation efficiency of about 60% to about 95%.

56. The process of any one of claims 47-55, comprising a loading level of about 2% to about 50%.

57. The process of any one of claims 47-56, wherein the plurality of nanoparticles have a release duration of about 7 days to about 180 days.

58. A process for generating a plurality of microgel particles, the process comprising:
(a) mixing a nanoparticle or polyelectrolyte nanocomplex (PNC) comprising one or more neuromodulators, a carrier molecule, and a counter ion polymer, and optionally a biodegradable polymer, with a hydrogel precursor;
(b) forming a hydrogel comprising the nanoparticle or polyelectrolyte nanocomplex (PNC) comprising one or more neuromodulators, a carrier molecule, and a counter ion polymer, and optionally a biodegradable polymer; and
(c) mechanically breaking the hydrogel into a plurality of microgel particles.

59. The process of claim 58, wherein the plurality of microgel particles has a nominal size ranging from about 10 μm to 1,000 μm.

60. A method for treating a disease or condition, the method comprising administering a nanoparticle of any of claims 1-14 or the microgel of any one of claims 15-31, to a subject in treat of treatment thereof.

61. The method of claim 60, wherein the disease or condition is selected from the group consisting of a cosmetic condition, focal dystonias, cervical dystonia (CD), chronic sialorrhea, and muscle spasticity.

62. The method of claim 61, wherein the muscle spasticity is related to an overactive muscle movement selected from the group consisting of cerebral palsy, post-stroke spasticity, post-spinal cord injury spasticity, spasms of the head and neck, eyelid, vagina, limbs, jaw, and vocal cords, clenching of muscles associated with muscles of the esophagus, jaw, lower urinary tract and bladder, and anus, and refractory overactive bladder.

63. The method of claim 60, wherein the disease or condition comprises muscle disorder selected from the group consisting of strabismus, blepharospasm, hemifacial spasm, infantile esotropia, restricted ankle motion due to lower-limb spasticity associated with stroke in adults, and lower-limb spasticity in pediatric patients two years of age and older.

64. The method of claim 60, wherein the disease or condition comprises excessive sweating.

65. The method of claim 60, wherein the disease or condition is selected from the group consisting of a headache, a migraine headache, neuropathic pain, chronic pain, osteoarthritis pain, arthritic pain, allergy symptoms, depression, and premature ejaculation.

66. The method of any one of claims 60-65, comprising administering two or more formulations of the nanoparticle or microgel, wherein the two or more formulations of the nanoparticle or microgel each have a different release profile.

67. A pharmaceutical composition comprising a nanoparticle of any of claims 1-14 or the microgel of any one of claims 15-31 and a pharmaceutically acceptable carrier.

68. A kit comprising a PNC or nanoparticle of any of claims 1-14 and/or a microgel of any one of claims 15-31.

69. A sustained release formulation comprising the PNC or nanoparticle of any of claims 1-14 or the microgel of any one of claims 15-31, wherein the formulation provides an effective concentration of the one or more neuromodulators in soft tissue for a period of time between about 3 days to about 200 days.

70. A method for treating a disease or condition, the method comprising administering a sustained release formulation comprising the PNC or nanoparticle of any of claims 1-14 or the microgel of any one of claims 15-31, the method comprising local administration by injection of the sustained release formulation, wherein the one or more neuromodulators is released from the sustained release formulation over a period of time from about 3 days to about 200 days, thereby treating a disease or condition with a measurable effect over 2 weeks to 40 weeks.

71. The method of claim 70, wherein the disease or condition is selected from the group consisting of a cosmetic condition, focal dystonias, cervical dystonia (CD), chronic sialorrhea, and muscle spasticity.
